SEQUENCE LISTING (6 sequences)

SEQ ID NO: 1 - protein, 5 aa, Artificial Sequence
  Delta receptor antagonists ICI-174,864.
YXXFL

SEQ ID NO: 2 - protein, 5 aa, Unknown
  Enkephalin
YGGFM

SEQ ID NO: 3 - protein, 5 aa, Unknown
  Enkephalin
YGGFL

SEQ ID NO: 4 - protein, 17 aa, Unknown
  Dynorphin
YGGFLRRIRPKLKWDNQ

SEQ ID NO: 5 - protein, 8 aa, Unknown
  AVP receptor antagonist
YFQNRPRY

SEQ ID NO: 6 - protein, 9 aa, Unknown
  AVP peptide
CYFQNCPRG

(19) United States
(12) Patent Application Publication
(10) Pub. No.: US 2010/0173926 A1
(43) Pub. Date: Jul. 8, 2010

(54) Methods for Regulating Neurotransmitter Systems by Inducing Counteradaptations

(76) Inventor: Alexander Michalow, Bourbonnais, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/627,081

(22) Filed: Nov. 30, 2009

Related U.S. Application Data
(63) Continuation of application No. 11/711,487, filed on Feb. 27, 2007, now abandoned.
Application No. 11/711,487 is a continuation-in-part of application No. 11/234,850, filed on Sep. 23, 2005.
(60) Provisional application No. 60/612,155, filed on Sep. 23, 2004.
(60) Provisional application No. 60/777,190, filed on Feb. 27, 2006.
(60) Provisional application No. 60/858,186, filed on Nov. 9, 2006.

(51) Int. Cl. A61K 31/439; A61P 25/04

(52) U.S. Cl. 514/279

(57) ABSTRACT

The present invention relates to methods for regulating neurotransmitter systems by inducing a counteradaptation response. According to one embodiment of the invention, a method for regulating a neurotransmitter includes the step of repeatedly administering a ligand for a receptor in the neurotransmitter system, with a ratio of administration half-life to period between administrations of no greater than ½. The methods of the present invention may be used to address a whole host of undesirable mental, neurological and physiological conditions.

### CROSS-REFERENCE TO RELATED APPLICATIONS

This application is a continuation-in-part under 35 U.S.C. §120 of U.S. patent application Ser. No. 11/234,850, filed on Sep. 23, 2006, which in turn claims the benefit under 35 U.S.C. §119(e) of U.S. Provisional Patent Application Ser. No. 60/612,155 entitled “COUNTER-ADAPTATION THERAPY FOR TREATMENT OF DEPRESSION AND OTHER MENTAL CONDITIONS,” and filed on Sep. 23, 2004. This application also claims priority to U.S. Provisional Patent Application Ser. No. 60/777,190, entitled “METHOD OF REGULATING THE CRF AND AVP SYSTEMS BY INDUCING COUNTERADAPTATIONS,” and filed on Feb. 27, 2006; and to U.S. Provisional Patent Application Ser. No. 60/858,186, entitled “OPIATE ANATOGONISTS FOR COUNTERADAPTATION THERAPY,” and filed on Nov. 9, 2006. The above-referenced provisional application is hereby incorporated herein by reference in its entirety.

### BACKGROUND OF THE INVENTION

1. Field of the Invention

The present invention relates generally to neurotransmitter systems. The present invention relates more particularly to methods for regulating these neurotransmitter systems by inducing counteradaptative responses.

2. Technical Background

Mood, mood disorders and related conditions are a result of a complex web of central nervous system events that interrelate many neurotransmitter systems. A most common mood disorder is depression. Depression is a clinical diagnosis with numerous somatic and mental symptoms, which is due to an alteration of numerous neurotransmitter systems. While the neurotransmitter systems most commonly related with depression are the norepinephrine and serotonin systems, current research indicates that other systems, such as the substance P system, the dynorphin system (kappa receptors), the endogenous endorphin system (mu and delta opiate receptors), the corticotropin releasing factor system, and the arginine vasopressin systems are also involved in depression. Further, these neurotransmitter systems are also related to a whole host of other undesirable mental, neurological and physiological conditions, including bipolar disorders, obsessive-compulsive disorders, anxiety, phobias, stress disorders, substance abuse, sexual disorders, eating disorders, motivational disorders, pain disorders, cardiovascular disorders, aging-related disorders, and immune-system related disorders.

Conventional strategies for treating neurotransmitter-linked conditions are centered on improving abnormally high or low levels of synaptic neurotransmitters. Conventional therapeutic agents work to directly regulate the functioning of the neurotransmitter systems. Such agents may be anxiolytic agents, hypnotic agents, or selective reuptake inhibitors, and include benzodiazepines (e.g., diazepam, lorazepam, alprazolam, temazepam, flurazepam, and chlodiazepoxide), TCAs, MAOIs, SSRIs (e.g., fluoxetine hydrochloride), NRIs, SNRIs, CRF modulating agents, serotonin pre-synaptic autoreceptor antagonists, 5HT₁ agonist, GABA-A modulating agents, serotonin 5H_{2C} and/or 5H_{2B} modulating agents, beta-3 adrenoceptor agonists, NMDA antagonists, V1B antagonists, GPCR modulating agents, dynorphin antagonists, and substance P antagonists.

Conventional therapeutic agents and methods, while somewhat effective, suffer from a few disadvantages. For example, use of many conventional therapeutic agents is attended by side effects, such as sexual dysfunction, nausea nervousness, fatigue, dry mouth, blurred vision and weight gain. Further, patients can adapt or build up a resistance to conventional therapeutic agents with repeated use, making them lose efficacy over time.

### SUMMARY OF THE INVENTION

One aspect of the present invention relates to a method of regulating a neurotransmitter system by inducing a counteradaptation in a patient, the neurotransmitter system including a type of receptor, the method comprising: repeatedly administering to the patient a ligand for the type of receptor, each administration having an administration half-life, thereby causing the ligand to bind receptors of that type during a first time period associated with each administration, thereby inducing, maintaining or improving a counteradaptation, wherein the counteradaptation causes the regulation of the neurotransmitter system, and wherein the ratio of the administration half-life to the period between administrations is no greater than ½.

In one aspect of the invention, the neurotransmitter system is the SP system; the type of receptor is SP receptors; the ligand is an SP receptor agonist; and the counteradaption causes a down-regulation of the SP system.

In another aspect of the invention, the neurotransmitter system is the endogenous endorphin system; the type of receptor is mu and/or delta opiate receptors; the ligand is a mu and/or delta opiate receptor agonist; and the counteradaption causes an up-regulation of the endogenous endorphin system.

In yet another aspect of the invention, the neurotransmitter system is the dynorphin system; the type of receptor is kappa receptors; the ligand is a kappa receptor agonist; and the counteradaption causes a down-regulation of the dynorphin system.

In still yet another aspect of the invention, the neurotransmitter system is the serotonin system; and the counteradaption causes an up-regulation of the serotonin system. Thus, in one embodiment of this aspect of the invention, the type of receptor is serotonin pre-synaptic autoreceptors; and the ligand is a serotonin pre-synaptic autoreceptor agonist. In another embodiment of this aspect of the invention the type of receptor is serotonin post-synaptic receptors; and the ligand is a serotonin post-synaptic autoreceptor antagonist.

In still yet another aspect of the invention, the neurotransmitter system is the norepinephrine system; and the counteradaption causes an up-regulation of the norepinephrine system. Thus, in one embodiment of this aspect of the invention, the type of receptor is norepinephrine pre-synaptic alpha-2 adrenergic receptors; and the ligand is a norepinephrine pre-synaptic alpha-2 adrenergic receptor agonist. In another embodiment of this aspect of the invention the type of receptor is norepinephrine post-synaptic adrenergic receptors; and the ligand is a norepinephrine post-synaptic adrenergic receptor antagonist.

In still yet another aspect of the invention, the neurotransmitter system is the CRF system; the type of receptor is CRF receptors; the ligand is a CRF receptor agonist; and the counteradaptation causes a down-regulation of the CRF system.

In still yet another aspect of the invention, the neurotransmitter system is the CRF system; the type of receptor is CRF receptors; the ligand is a CRF receptor antagonist; and the counteradaptation causes a up-regulation of the CRF system.

In still yet another aspect of the invention, the neurotransmitter system is the AVP system; the type of receptor is AVP receptors; the ligand is a AVP receptor agonist; and the counteradaptation causes a down-regulation of the AVP system.

In still yet another aspect of the invention, the neurotransmitter system is the AVP system; the type of receptor is AVP receptors; the ligand is a AVP receptor antagonist; and the counteradaptation causes a up-regulation of the AVP system.

In yet another embodiment of the invention, a method is provided for inducing a regulation of a neurotransmitter system, the neurotransmitter system including a type of receptors linked to an undesirable mental, neurological or physiological condition. The method comprising the step of: repeatedly administering to the patient a ligand for the type of receptor, each administration having an administration half-life, thereby causing the ligand to bind a substantial fraction of receptors of that type during a first time period associated with each administration, thereby inducing a counteradaptation; wherein the counteradaptation causes the regulation of the neurotransmitter system during a second time period associated with each administration, the second time period being subsequent to the first time period.

In yet another aspect of the present invention, the methods described herein are used to address or treat an undesirable mental, neurological or physiological condition in a patient, the undesirable mental, neurological or physiological condition being linked to receptors of the type of receptor.

In yet another aspect of the present invention, the methods described herein are used to address or treat an undesirable immune system-related condition in a patient in need thereof, the immune-system related condition being linked to receptors of the type of receptors, the method resulting in an up-regulation of the immune system.

In yet another aspect of the present invention, the methods described herein are used to address or treat a cariovascular or lipid or cholesterol metabolism-related condition in a patient in need thereof, the cariovascular or lipid or cholesterol metabolism-related condition being linked to receptors of the type of receptor.

In yet another aspect of the present invention, the methods described herein are used to address or treat insufficient preparedness for a future athletic activity.

In yet another aspect of the present invention, the methods described herein are used to address or treat an undesirable condition linked to the Sirt1 pathway.

The methods of the present invention result in a number of advantages over prior art methods. For example, the methods of the present invention can be used to address a whole host of undesirable mental, neurological and physiological conditions with reduced side effects. In certain embodiments of the invention, the desired therapeutic benefit can be timed to coincide with a desired time of day or task to be performed by the patient.

Additional features and advantages of the invention will be set forth in the detailed description which follows, and in part will be readily apparent to those skilled in the art from the description or recognized by practicing the invention as described in the written description and claims hereof, as well as in the appended drawings.

It is to be understood that both the foregoing general description and the following detailed description are merely exemplary of the invention, and are intended to provide an overview or framework for understanding the nature and character of the invention as it is claimed.

The accompanying drawings are included to provide a further understanding of the invention, and are incorporated in and constitute a part of this specification. The drawings are not necessarily to scale, and sizes of various elements may be distorted for clarity. The drawings illustrate one or more embodiment(s) of the invention, and together with the description serve to explain the principles and operation of the invention.

### BRIEF DESCRIPTION OF THE DRAWINGS

FIG. 1 is a graph of in vivo ligand concentration (part a) and mood vs. time (part b) according to one embodiment of the invention;

FIG. 2 is a graph of mood vs. time for several administrations of a ligand according to another embodiment of the invention;

FIG. 3 is a graph of in vivo ligand concentration vs. time for the administration via a single injection of a ligand with a relatively long compound half-life;

FIG. 4 is a graph of in vivo ligand concentration vs. time for the administration via time-release transdermal patch of a ligand with a relatively short compound half-life;

FIG. 5 is a graph of in vivo ligand concentration vs. time for the administration via time-release transdermal patch of a ligand with a relatively short compound half-life, when the patch is removed during the administration; and

FIG. 6 is a graph of in vivo ligand concentration (part a) and mood vs. time (part b) according to another embodiment of the invention.

### DETAILED DESCRIPTION OF THE INVENTION

The present invention relates generally to the regulation of neurotransmitter systems by exploiting the patient's response to a pharmaceutical agent (a “counteradaptation”), rather than by relying on the direct effect of the agent for an improved clinical effect. In general, pharmaceutical agents are chosen so that the counteradaptation is beneficial to the patient and eventually provides the desired long-term effect. The methods of the present invention differ from conventional methods in that the direct effect of the agent is a modulation of neurotransmitter receptors that is generally associated with a worsening of symptoms. In response to the direct effect of the agent, however, the brain responds by a counteradaptation, resulting in the desired regulation of the neurotransmitter system when any direct effect of the agent wears off. The regulation may be any change in neurotransmitter system functioning, and may be, for example, an up-regulation or a down-regulation. A specific acute response is induced directly in order to generate a desired long-term effect indirectly. In a simple analogy, just as euphoria-stimulating agents such as morphine and cocaine result in depression upon their withdrawal, dysphoria-stimulating agents result in “anti-depression” upon their withdrawal.

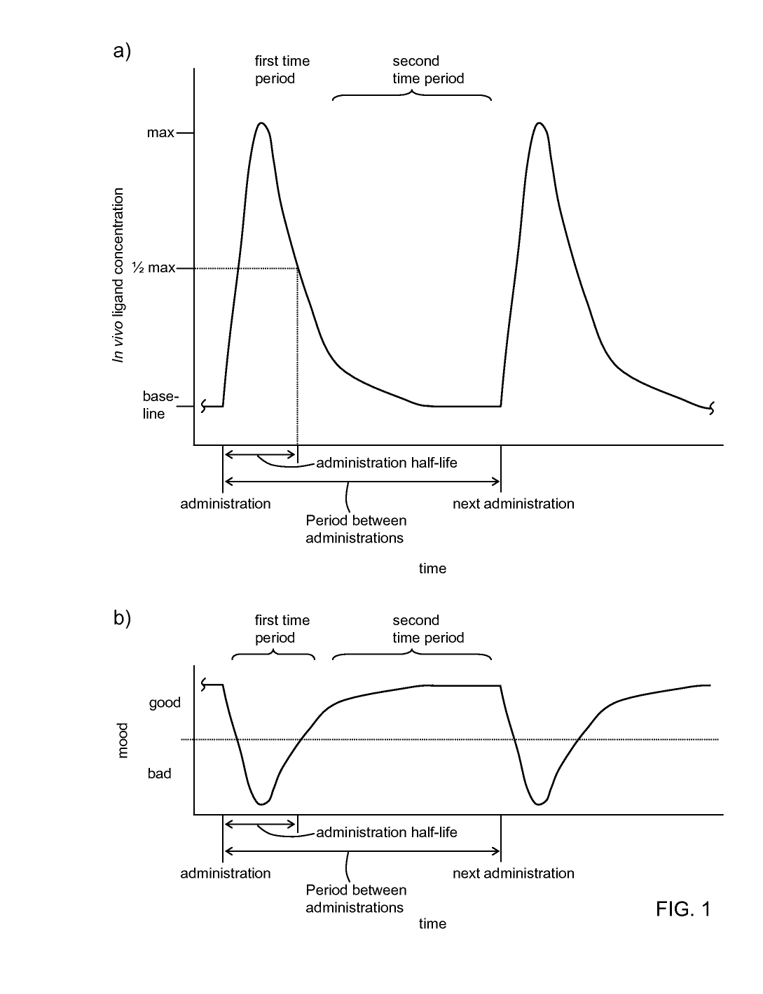

One embodiment of the present invention relates to a method of a regulating a neurotransmitter system. Generally, a neurotransmitter system is a system of natural neurotransmitter compounds and synaptic receptors that participates in central nervous system signal transmission. The neurotransmitter system includes a type of receptors. The type of receptors may be, for example, linked to an undesirable mental, neurological or physiological condition. FIG. 1 includes a graph of in vivo ligand concentration versus time for a method according to one embodiment of the invention. As illustrated in FIG. 1, the method includes the step of repeatedly administering to a patient a ligand for the type of receptor, thereby causing the ligand to bind receptors of that type during a first time period associated with each administration. As used herein, a ligand is a compound that binds to (e.g., interacts with in either a covalent or non-covalent fashion) receptors of the type of receptor, and may be, for example, an agonist for the receptor or an antagonist for the receptor. The binding of the ligand to the receptors induces a counteradaptation, which causes the regulation of the neurotransmitter system. FIG. 1 shows a couple of administrations of ligand occurring in the middle of the method, and not the first couple of administrations. Each administration is a single cycle in which the in vivo concentration of the ligand begins at a baseline level, goes up to a maximum level, and drops back down to the baseline level. The graph of FIG. 1 shows two such administrations. Depending on the dosing regimen, each administration of the ligand may be performed, for example, by giving the patient a single unit dose (e.g., pill, capsule) or injection; multiple unit doses or injections; or continuously (e.g., intravenous or slow-release patch).

Examples of types of neurotransmitter systems and types of receptors with which the method may be practiced include the Substance P system, in which the type of receptors may be NK-1, NK-2 and/or NK-3 receptors; the endogenous endorphin system in which the type of receptors may be mu and/or delta opiate receptors; the dynorphin system in which the type of receptors may be kappa receptors; the serotonin system in which the type of receptors may be inhibitory serotonin pre-synaptic autoreceptors (e.g., 5HT_{1A} and/or 5HT_{1B} autoreceptors) and/or serotonin post-synaptic receptors (e.g. 5HT₁, 5HT₂, 5HT₃, 5HT₄, 5HT₅, 5HT₆ and/or 5HT₇ receptors); the norepinephrin system in which the type of receptors may be inhibitory norepinephrine pre-synaptic alpha-2 adrenergic receptors and/or norepinephrine post-synaptic adrenergic receptors; the corticotropin releasing factor (CRF) system, in which the type of receptors may be CRF receptors (e.g. CRF-1 receptors and/or CRF-2 receptors; and the arginine vasopressin (AVP) system, in which the type of receptors may be AVP rececptors (e.g., V1R, also known as V1a, V2R and/or V3R, also known as V1b). These neurotransmitter systems and receptor types are linked to various undesirable mental, neurological and physiological conditions, as would be appreciated by the skilled artisan.

In certain aspects of the invention, an undesirable mental, neurological or physiological condition is linked to a type of receptor in the neurotransmitter system. If the undesirable mental, neurological or physiological condition is exacerbated by the binding of the receptor to its natural neurotransmitter, then it is said to be “positively linked” to that type of receptor. Conversely, if the undesirable mental, neurological or physiological condition is improved by the binding of the receptor to its natural neurotransmitter, then it is “negatively linked” to that type of receptor. For example, the undesirable mental, neurological or physiological condition of depression is negatively linked to serotonin post-synaptic receptors, because binding of these receptors to their natural neurotransmitter serotonin results in a decrease in the depression. The undesirable mental, neurological or physiological condition of depression is positively linked to kappa receptors, because binding of these receptors to their natural neurotransmitter dynorphin results in an increase in the depression.

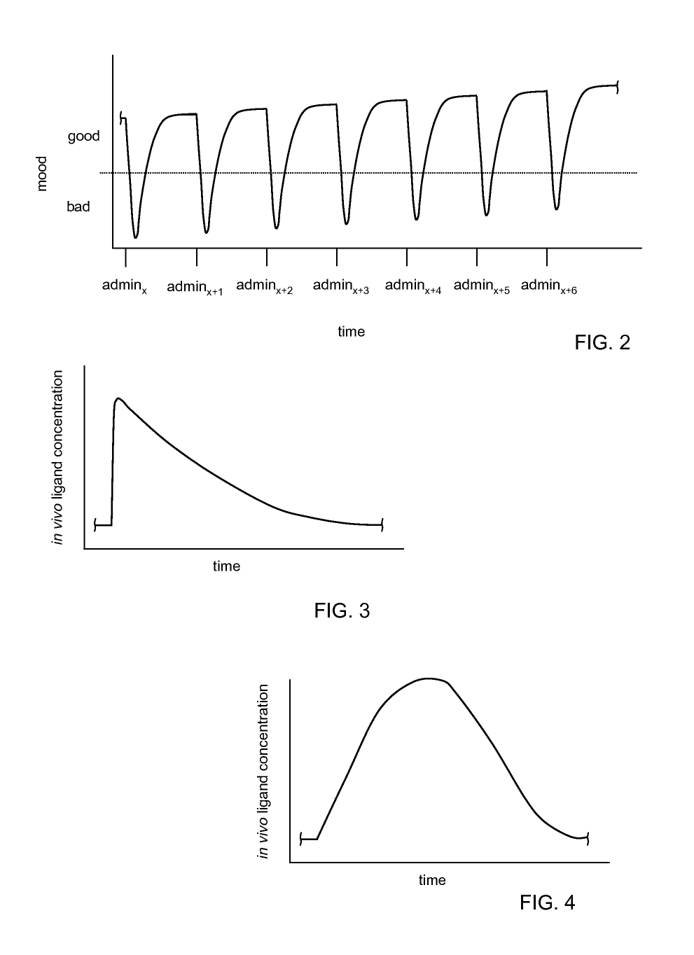

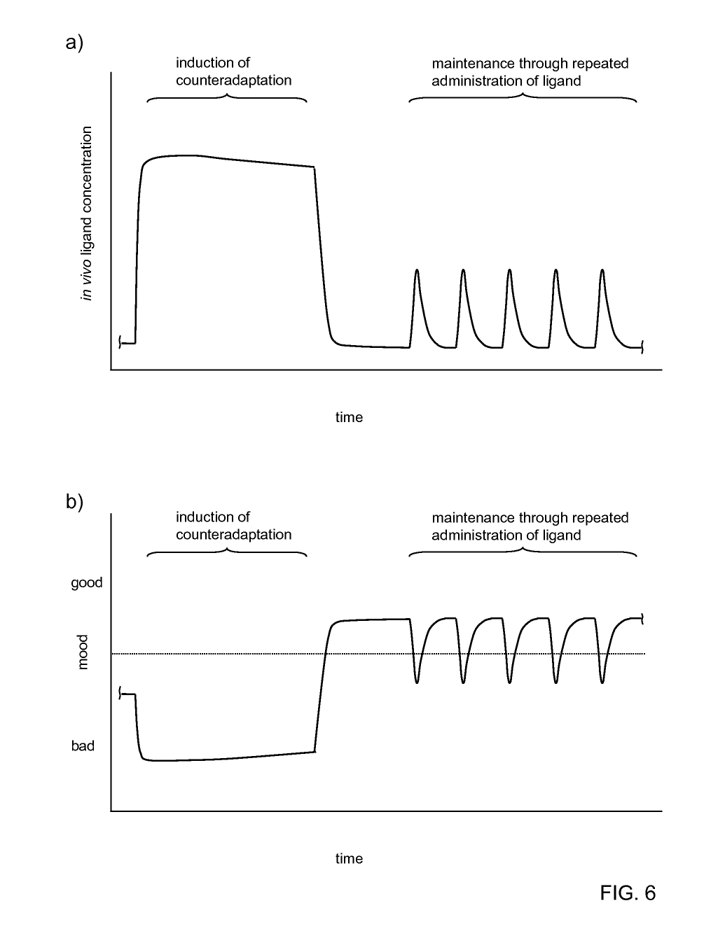

Instead of relying on the direct effect of ligand-receptor binding to regulate the neurotransmitter system, the methods of the present invention exploit the indirect counteradaptive effect to enhance or suppress neurotransmitter systems. The counteradaptation is the brain's natural response to the binding of the ligand. The initial effect of ligand binding may be a worsening of an undesirable mental, neurological or physiological condition linked to the neurotransmitter system. However, because the effects of the counteradaptation last long after the ligand is removed from the system, and can build up over repeated administration of the ligand, the counteradaptation causes an overall desirable regulation of the neurotransmitter system. The regulation of the neurotransmitter system can, in turn, provide a therapeutic benefit with respect to the undesirable mental, neurological or physiological condition. The regulation of the neurotransmitter system may be, for example, an increase in the counteradaptive response (as shown in FIG. 2, described below), or a maintenance of an already-induced counteradaptive response (as shown in FIG. 6, described below).

Counteradaptations are a manner by which the central nervous system maintains homeostasis. The counteradaptation is a result of the body's attempt to regulate the neurotransmitter system to its original steady-state level in order to prevent its over- or under-stimulation. Natural neurotransmitters bind with their receptors for only a short time, and are removed almost immediately from the synapse, and therefore do not cause a counteradaptive response. When a ligand interacts with a receptor for a longer period of time (e.g., because the ligand has a longer binding time or is continuously administered), however, cellular mechanisms gradually occur at the receptor/neurotransmitter level that act to counteract the direct effects of the ligand-receptor binding (i.e., the counteradaptation). The counteradaptation may be, for example, a change in the biosynthesis or release of a natural neurotransmitter that binds to the type of receptor, a change in the reuptake of a natural neurotransmitter that binds to the type of receptor, a change in the number of the type of receptors and/or binding sites on receptors of the type of receptor, a change in the sensitivity of receptors of the type of receptor to binding by the natural neurotransmitter and/or receptor agonists, or any combination thereof. Chronic use of a ligand thus induces (i.e., causes) a counteradaptation by stimulating processes that oppose the initial effects of the ligand, which over time results in a decrease in the effect of ligand-receptor binding.

When the ligand is a receptor agonist, the counteradaptation works to reduce the functioning of the neurotransmitter system (i.e., a “down-regulation”). The down-regulation may occur through, for example, a decrease in the biosynthesis or release of a natural neurotransmitter that binds to the type of receptor, an increase in the reuptake of a natural neurotransmitter that binds to the type of receptor, a decrease in the number of the type of receptors and/or binding sites on receptors of the type of receptor, a decrease in the sensitivity of receptors of the type of receptor to binding by the natural neurotransmitter and/or receptor agonists, or any combination thereof. Any of the above-recited counteradaptive responses will work to reduce the functioning of the neurotransmitter system, and can therefore provide a therapeutic benefit with respect to an undesirable mental, neurological or physiological condition that is positively linked to the neurotransmitter system.

Conversely, when the ligand is a receptor antagonist, the counteradaptation works to increase the functioning of the neurotransmitter system (i.e., an “up-regulation”). The up-regulation may occur through, for example, an increase in the biosynthesis or release of a natural neurotransmitter that binds to the type of receptor, a decrease in the reuptake of a natural neurotransmitter that binds to the type of receptor, an increase in the number of the type of receptors and/or binding sites on receptors of the type of receptor, an increase in the sensitivity of receptors of the type of receptor to binding by the natural neurotransmitter and/or receptor agonists, or any combination thereof. Any of the above-recited counteradaptive responses will work to increase the functioning of the neurotransmitter system, and therefore provide a therapeutic benefit with respect to an undesirable mental, neurological or physiological condition that is negatively linked to the neurotransmitter system.

Receptors in the brain are commonly regulated by a pre-synaptic negative inhibition control loop. Thus, for mood-elevating post-synaptic receptors (i.e., receptors negatively linked to an undesirable mental, neurological or physiological condition), it is desirable to use repeated agonist treatment at the associated inhibitory pre-synaptic receptors. Repeated agonist administration at a pre-synaptic inhibitory receptor results in a down-regulation of that receptor, lessening its inhibitory response and thereby increasing neural firing at the mood-elevating post-synaptic receptors and providing an elevation of mood.

An opposite strategy is desired for use with mood-depressing post-synaptic receptors (i.e., receptors positively linked to an undesirable mental, neurological or physiological condition). For such receptors, it is desirable to use repeated antagonist treatment at the associated inhibitory pre-synaptic receptors. Repeated antagonist administration at a pre-synaptic inhibitory receptor results in an up-regulation of that receptor, lessening its inhibitory response and thereby decreasing neural firing at the mood-depressing post-synaptic receptors and providing an elevation in mood.

The direct effect of the ligand binding during the first time period will often be an initial exacerbation of an undesirable mental, neurological or physiological condition linked to the type of receptor. For example, when the administered ligand is an antagonist for a type of receptor linked negatively to an undesirable mental, neurological or physiological condition, the short-term effect of the binding is to block the receptors and prevent them from binding the natural neurotransmitter and firing. Similarly, when the administered ligand is an agonist for a type of receptor positively linked to an undesirable mental, neurological or physiological condition, the short term affect of the binding is to cause the receptors to fire. Both the firing of receptors positively linked to the undesirable mental, neurological or physiological condition and the prevention of the firing of receptors negatively linked to the undesirable mental, neurological or physiological condition can cause an initial worsening of symptoms. When the short-term effect of ligand-receptor binding wears off (e.g., due to the removal of ligand from the system), the counteradaptation remains to provide the desired regulation of the neurotransmitter system. Repeated administration can cause a gradually increasing regulation of neurotransmitter systems. In certain embodiments of the present invention described below, measures are taken to limit the effect on the patient of the direct effect of ligand-receptor binding.

FIG. 1 also includes in part (b) a graph of mood vs. time for administration of an appropriate ligand for a mood-associated receptor. As shown in the example of FIG. 1, the direct effect of ligand administration may be a worsening of mood during each first time period. This worsening of mood tapers off as the in vivo concentration of the ligand falls to its steady state level. After the ligand concentration returns to its low steady state level, the counteradaptation remains in place to provide an overall improvement in mood during a second time period associated with each administration and subsequent to the first time period. FIG. 2 is a graph of mood vs. time for several administrations of a ligand during a method according to the present invention. As evidenced in FIG. 2 by the ever-increasing mood (i.e., the graph generally slants up with time), the strength of the counteradaptation may build up with time, with each administration causing additional counteradaptive response. As such, an increasing therapeutic benefit may be realized with repeated intermittent administration of the ligand. While FIGS. 1 and 2 depict an example in which the counteradaptation causes a net increase in mood, the skilled artisan will recognize that the methods described herein will be useful to treat any number of neurotransmitter-linked conditions, including those described in more detail hereinbelow.

Each administration of the ligand has an administration half-life. As shown in the graph of part (a) of FIG. 1, the in vivo concentration of the ligand is at a relatively low baseline level at the beginning of the administration (e.g., the swallowing of a pill, the application of a transdermal patch, or the beginning of intravenous administration), then rises to some maximum level. After reaching a maximum, the in vivo concentration of the ligand will decrease back down to the baseline level (e.g., due to metabolism/excretion of the ligand), where it remains until the next administration. As shown in FIG. 1, the administration half-life is measured as the period of time between the beginning of the administration and the half-maximum point of the in vivo concentration as the concentration drops from its maximum level to the baseline level.

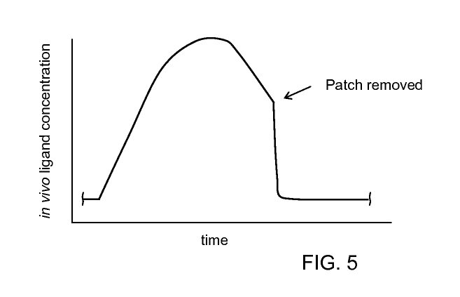

The administration half-life will be a function of the compound half-life (i.e., the half-life in vivo of the ligand compound itself) as well as of the route of administration. For example, FIG. 3 is a graph of in vivo concentration versus time for a single administration via injection of a ligand with a relatively long compound half-life. Because the injection gets the ligand into the bloodstream very quickly, the administration half-life approximates the compound half-life. In the example of FIG. 4, a ligand with a much shorter compound half life (e.g., a peptide) is administered using a time-release transdermal patch. Here, the concentration rises more slowly to a steady state maximum concentration, then falls off as the patch becomes depleted. Were the patch removed before depletion, the in vivo concentration would decrease rapidly down to the baseline level, as shown in FIG. 5. The administration half-life may be, for example, less than about a week, less than about three days, or less than about a day. More desirably, the administration half-life is less than about sixteen hours; less than about twelve hours, less than about eight hours; or less than about four hours. In certain embodiments of the invention, especially those using a ligand having a relatively long compound half-life, the administration half-life may be greater than about four hours; greater than about twelve hours; greater than about sixteen hours; or greater than about thirty hours.

The ligand has a compound half-life, defined as the in vivo half life of the ligand and its active metabolites (i.e., metabolites that are active at receptors of the type of receptor), divorced from any effects due to the route of administration. In certain embodiments of the present invention, it may be desirable to use a compound with a relatively short compound half-life. For example, in certain embodiments of the invention the compound half-life is less than about a week, less than about three days, or less than about a day. More desirably, the compound half-life is less than about sixteen hours; less than about twelve hours, less than about eight hours; or less than about four hours; or less than one hour. Some ligands, however, have relatively longer compound half-lives. For example, in certain embodiments of the invention, the compound half-life of the ligand is greater than about four hours; greater than about twelve hours; greater than about sixteen hours; or greater than about thirty hours.

The period between administrations is desirably selected so as to maximize the counteradaptive response to the ligand while maintaining an acceptably low and tolerable direct effect of ligand-receptor binding. For example, the administration of the ligand may be performed daily. In other embodiments of the invention, the period between administrations is two days or greater; three days or greater; five days or greater; one week or greater; two weeks or greater; or one month or greater. Similarly, the dose of the ligand at each administration is selected to be sufficient to trigger a counteradaptive response, but low enough that direct effects of ligand-receptor binding are low and tolerable to the patient.

When using a ligand having a compound half-life greater than about twelve hours, in order to increase the counteradaptation it may be desirable to repeatedly administer a second ligand for the type of receptor, with each administration of the second ligand having an administration half-life of less than about eight hours. In an example of a method according to the present invention, a ligand having a twenty-four hour compound half-life is administered every three days with a twenty four hour administration half-life, and a second ligand is administered daily with a six hour administration half-life. In such cases, when the ligand is a receptor agonist, the second ligand is desirably a receptor agonist; and when the ligand is a receptor antagonist, the second ligand is desirably a receptor antagonist.

The ratio of administration half-life to the period between administrations is desirably selected to maximize the counteradaptation while keeping any direct effects of ligand binding during the first time period at a low and tolerable level. According to one embodiment of the invention, the ratio of the administration half-life to the period between administrations is no greater than ½. Desirably, the ratio of the administration half-life to the period between administrations is no greater than ⅓. In certain embodiments of the invention, the ratio of the administration half-life to the period between administrations is no greater than ⅕; no greater than ⅛; or no greater than 1/12. It may be, however, desirable to administer the ligand relatively often, in order to maintain a desired level of counteradaptation. For example, in certain desirable embodiments of the invention the ratio of administration half-life to the period between administrations may be greater than 1/100; greater than 1/50; greater than 1/24; greater than 1/12; greater than ⅛; greater than ⅕; greater than ¼; or greater than ⅓.

A substantial fraction of the receptors of the type of receptor are desirably bound to the ligand during the first time period associated with each administration, so as to cause a counteradaptation to the ligand binding. For example, at least about 30%, at least about 50%, at least about 75%, or at least about 90% of the receptors of the type of receptor are desirably bound by the ligand during each first time period.

Similarly, the first time period associated with each administration is desirably long enough to cause a substantial counteradaptation. For example, each first time period is desirably at least about five minutes in duration; at least about thirty minutes in duration; at least about an hour in duration; at least about two hours in duration; or at least about four hours in duration. In certain desirable embodiments of the invention, each first time period is about eight hours in duration. However, in cases where the direct effect of ligand binding is a noticeable worsening in an undesirable condition, it may be desirable to maintain the first time period no longer than necessary to get an acceptable level of counteradaptation. For example, in certain embodiments of the invention the first time period is desirably less than about twenty four hours in duration; less than about sixteen hours in duration; less than about twelve hours in duration; less than about eight hours in duration; or less than about six hours in duration.

In desired embodiments of the invention, a substantial fraction of the receptors remain unbound to the ligand during a second time period associated with each administration and subsequent to the first time period. A low level of ligand-receptor binding allows the patient to enjoy the effects (e.g., the therapeutic benefit) of the counteradaptation without interference from any ill effects of direct ligand binding. For example, desirably no more than about 50%, no more than about 25%, no more than about 10% of the receptors are bound to the ligand during each second time period.

The second time period associated with each administration is the time during which a substantial fraction of the receptors of the type of receptor are unbound to the ligand. During each second time period, the patient may enjoy any therapeutic benefit of the counteradaptation, as no direct ligand-receptor binding effects would remain. As such, each second time period is desirably as long as possible. For example, each second time period is desirably at least about two hours in duration; at least about ten hours in duration; or at least about fifteen hours in duration. However, it may be desirable to keep each second time period relatively short, in order to decrease the period between administrations thereby increase the counteradaptation. For example, in certain embodiments of the invention each second time period is desirably no more than about twenty hours in duration; no more than about thirty hours in duration; or no more than about fifty hours in duration.

In order to build up a counteradaptation over time and to minimize any initial exacerbation of an undesirable mental, neurological or physiological condition, it may be desirable to begin the treatment with a relatively low dose of ligand at each administration, and increase the dosage over time. Increasing dosages can also be used to account for any tolerance the patient builds up to the ligand. For the sake of convenience, it may be desirable to increase the dosage intermittently over time (i.e., increase the dosage with a period longer than the period between administrations). For example, in certain embodiments of the invention the dosage is increased with a period between increases of no less than a week; no less than two weeks; no less than three weeks; no less than a month; no less than two months; no less than three months; no less than six months, or no less than one year. At each increase in dosage, the dose is desirably increased by at least about 5%; at least about 10%; at least about 25%; at least about 50%; or at least about 100% of the initial dose. It may, however, be desirable to maintain the maximum dosage within certain limits. For example, in certain embodiments of the invention the maximum dosage may be within three hundred times the initial dosage, within one hundred times the initial dosage, within fifty times the initial dosage, or within twenty times the initial dosage.

In one example of a dosing schedule, low doses of a ligand are given for one, two, or three weeks. These initial doses are high enough to induce a counteradaptive response, but low enough to cause only minimal direct effects due to ligand-receptor binding. The dose is then increased. The increase may be as small as 10%; for more rapid induction of a counteradaptive response, however, it is desirable to at least double the initial dose. After four to six weeks the dosage is again increased. This pattern is followed every one, two, four or six months. The endpoint for the maximum dosage will depend on individual tolerance to the ligand and the development of side effects and direct effects from the larger doses.

To reduce the impact of any direct effects of ligand-receptor binding, it may be desirable to time the administration of the ligand so that the first time period occurs during a time when adverse effects on the patient will be minimized. The patient will not notice many of the direct effects of ligand-receptor binding (e.g., a decrease in mood) if she is asleep. For example, it may be desirable to time the administration of the ligand so that a substantial fraction of the first time period occurs while the patient is asleep, so that any direct effects of ligand-receptor binding are not noticed. For example, at least 40%; at least 60%; or at least 85% of the first time period desirably occurs while the patient is asleep. In order to achieve such timing, it may be desirable to perform a substantial fraction of the administrations of the ligand within the hour before the patient goes to bed. For example, desirably at least 50%; at least 75%; at least 90%; or at least 95% of the administrations of the ligand are performed within the hour before the patient goes to bed.

There is no contraindication for daytime administration, however, and in other embodiments of the invention, each administration of the ligand is performed more than one hour before the patient goes to bed. In one example of a method according to the present invention, a patient who has been administered a ligand daily for two or three months and has developed a counteradaptation and, for example, some associated improvement in mood. If there were a particular time of day the patient wanted to enhance daytime mood, the time of ligand administration could be moved so that the desired time would fall within the second time period associated with that administration. If the patient wanted an elevated mood at 6 p.m., he could administer an appropriate ligand (e.g., naloxone, a mu and/or delta opiate receptor antagonist with a compound half-life of one hour) at 2 p.m. The direct effect of naloxone-receptor binding (a bad mood) would last only a couple of hours, leaving only the good mood caused by the counteradaptation by 6 p.m.

The administration of the ligand is desirably repeated enough times to build up a suitably large counteradaptive effect. As such, in the methods of the present invention, the administration is desirably performed at least five times, at least ten times, at least twenty-five times, or at least fifty times.

Each administration of the ligand may be performed orally, transdermally, through inhalation, subcutaneously, intravenously, intramuscularly, intraspinally, intrathecally, transmucosally, or using an osmotic pump, a microcapsule, an implant or a suspension. The skilled artisan will select the route of administration based upon the identity of the ligand, its compound half-life, the desired dose and the desired administration half-life.

It may be desirable to administer the ligand using both a rapidly absorbed loading dose (in order to get a fast ligand-receptor binding), and a gradually absorbed dose (in order to maintain a desired level of ligand-receptor binding over the desired length of the first time period). A rectal suppository having a rapidly-absorbing outer covering and a more slowly absorbing center could be used for such an administration. Alternatively, the loading dose could be given sublingually, and the gradually absorbed dose could be given transdermally via patch.

A carrier in the blood may be used to increase the administration half-life of the ligand once it is in circulation. For example, U.S. Pat. Nos. 6,610,825 and 6,602,981, each of which is incorporated herein by reference in its entirety, describe a method by which ligands are bound to blood cells or proteins in order to extend their administration half-life. Adessi et al (Curr Med Chem, 9(9); May, 2002; 963-978) describe a method by which to stabilize peptide ligands.

The methods of the present invention may be used to treat or address any undesirable condition linked to the neurotransmitter system. Examples of such conditions include chronic pain, mood disorders, eating disorders, anxiety disorders, motivational and performance problems, inflammatory conditions, nausea, emesis, urinary incontinence, skin rashes, erythema, eruptions, cardiovascular conditions, immune system-related conditions, and effects of aging. More examples of undesirable mental, neurological or physiological conditions are described below.

It may also be desirable to administer an anxiolytic agent in combination with the ligand, so as to reduce any direct effects of ligand-receptor binding. The anxiolytic agent may especially help mitigate the effects of ligand-receptor binding on the patient's sleep. The anxiolytic agent may, for example, affect a GABA pathway. The anxiolytic agent may be, for example, a benzodiazepine such as diazepam, lorazepam, alprazolam, temazepam, flurazepam, and chlodiazepoxide. Similarly, it may be desirable to administer a hypnotic agent or a selective serotonin reuptake inhibitor in combination with the ligand, so as to reduce any direct effects of ligand-receptor binding. Each of these agents may be administered at the same time as the ligand, or at a different time. It may also be desirable to add tryptophan to the patient's diet, as described in U.S. Pat. Nos. 4,377,595 and 5,958,429, each of which is incorporated herein by reference in its entirety.

In some cases, one direct effect of ligand-receptor binding is a decrease in immune system function. Accordingly, it may be desirable to administer an autoimmune medication in combination with the ligand during the first time period. Examples of suitable autoimmune therapy include medications such as corticosteroids, chlorambucil, cyclosporine, cyclophosphamide, methotrexatate, azathioprine, TNFα antagonists, and therapies such as systemic enzyme therapy, gene therapy and irradiation therapy. Of course, as the skilled artisan will realize, other autoimmune medications may be used in the methods of the present invention, including those developed in the future. In certain embodiments of the invention, autoimmune therapy is not administered during the second time period.

Similarly, it may be desirable to administer an antiviral agent in combination with the ligand during the first time period. Examples of suitable antiviral agents include interferon, ribavirin, protease inhibitors, amantadine, rimantadine, pleconaril, antibodies (monoclonal, anti-VAP, receptor anti-idiotypic, extraneous receptor and synthetic receptor mimics), acyclovir, zidovudine (AZT), lamivudine, RNAase H inhibitors, integrase inhibitors, attachment blockers of transcription factors to viral DNA, so-called ‘antisense’ molecules, synthetic ribozymes, zanamivir, and osletamivir. Of course, as the skilled artisan will realize, other antiviral medications may be used in the methods of the present invention, including those developed in the future. In certain embodiments of the invention, the antiviral agent is not administered during the second time period.

Similarly, it may be desirable to administer an antimicrobial agent, an antifungal agent, and/or an antineoplastic agent in combination with the ligand during the first time period. In certain embodiments of the invention, the antimicrobial agent, the antifungal agent, and/or the antineoplastic agent is not administered during the second time period.

It may be desirable to administer an anti-cancer agent in combination with the ligand during the first time period. Suitable anti-cancer agents include, for example, adriamycin, busulfan, carboplatin, chlorambucil, cisplatin, cyclophosphamide, ifosfamide, mechlorethamine, melphalan, procarbazine, temozolamide, daunorubicin, doxorubicin, idarubicin, bleomycin, mitomycin, mitoxantrone, plicamycin, cytarabine fluorouracil, hydroxyurea, methotrexate, asparaginase, pegaspargase, irinotecan, topotecan, bicalutamide, estramustine, flutamide, leuprolide, megestrol, nilutamide, testosterone, triptorelin, anastrazole, letrozole, aldesleukin, alemtuzumab, gemtuzumab, toremifene, trastuzumab, etoposide, docetaxel, paclitaxel, vinblastine, vincristine, vinorelbine, altretamine, Erlotinib, gleevec, curcumin, tamoxifen, bortezomib, gefitinib, imatinib, cancer cell growth inhibitors derived from 3,4-methylenedioxy-5,4′-dimethoxy-3′-amino-Z-stilbene, hydroxyphenstatin and its sodium diphosphate prodrug, histone deacetylase inhibitors, suberoylanilide hydroxamic acid, trichostatin A, sodium butyrate, metformin, five-lipoxygenase (5-LO) antagonists, antisense oligonucleotides targeting the RIα regulatory subunit of protein kinase A type I, Vitamin E and its analogs, vitamin E succinate (VES), and gene therapy. Of course, as the skilled artisan will realize, other autoimmune medications may be used in the methods of the present invention, including those developed in the future. In certain embodiments of the invention, the antiviral agent is not administered during the second time period.

It may be desirable to administer conventional pharmaceutical agents in combination (e.g., simultaneously or sequentially) with the ligand. Administration of such an agent is especially desirable when it is an agonist for a type of receptor that has been increased in number and/or sensitivity through a counteradaptation, or is an antagonist for a type of receptor that has been decreased in number and/or sensitivity through a counteradaptation. Examples of conventional pharmaceutical agents that may administered in combination with the ligand include TCAs, MAOIs, SSRIs, NRIs, SNRIs, CRF modulating agents, serotonin pre-synaptic autoreceptor antagonists, 5HT₁ agonist, dynorphin antagonists, GABA-A modulating agents, serotonin 5H_{2C} and/or 5H_{2B} modulating agents, beta-3 adrenoceptor agonists, NMDA antagonists, V1B antagonists, GPCR modulating agents, or substance P antagonists. Desirably, the additional pharmaceutical agent has a relatively short administration half-life, so that it can be administered during the second time period, with its effect substantially absent by the next administration of the ligand. Such an administration regimen maintains a high level of counteradaptation, while maximizing the effect of the pharmaceutical agent during the second time period.

It may also be desirable to take advantage of direct binding of the receptors to provide a desired clinical effect. For example, when the ligand is a receptor agonist, it may be desirable to administer an antagonist for the type of receptor during one or more of the second time periods associated with each administration and subsequent to the first time period. However, the antagonist for the type of receptor is desirably not administered during the first time period associated with each administration. Similarly, when the ligand is a receptor antagonist, it may be desirable to administer an agonist for the type of receptor during one or more of the second time periods associated with each administration and subsequent to the first time period. However, the agonist for the type of receptor is desirably not administered during the first time period associated with each administration. Preferably the antagonist has an in vivo half life of less than 12 hours, less than 8 hours, or less than 6 hours, such that it would not interfere with the subsequent administration of the agonist.

Another embodiment of the present invention is illustrated by the graphs of in vivo ligand concentration (part a) and mood vs. time (part b) of FIG. 6. In this method, a counteradaptation is first induced by giving the patient one or more doses of a ligand for the type of receptor. As shown in FIG. 6, this could be through repeated or continuous administration of high doses of the ligand. Relatively high, long-term doses of the ligand will induce a strong counteradaptive effect, but may cause the patient to suffer marked direct effects from ligand-receptor binding, as shown in the graph of mood vs. time of FIG. 6. In such cases, it may be desirable to keep the patient hospitalized during the initial induction of the counteradaptive response. After the counteradaptive response is induced, it is maintained repeatedly administering the ligand to the patient with a ratio of administration half-life to period between administrations no greater than ½. The repeated administration may be performed substantially as described above.

Through regulation of the function of neurotransmitter systems, the methods of the present invention may be used to improve undesirable mental, neurological and physiological conditions, even if they are not able to cure them. The methods of the present invention may make undesirable mental, neurological, and physiolocigal conditions more amenable to conventional therapies. For example, even if clinical depression is not cured, the improved mood caused by the use of the methods of the present invention may help improve the depression. As described above, the use of conventional antidepressants may also be made more efficacious. In another example, even if cancer is not cured, the regulation of the neurotransmitter acts to suppress tumor growth and/or metastasis, and may make conventional cancer therapies and/or the immune system better able to eliminate the cancerous growth. The therapeutic benefits caused by the regulation of the neurotransmitter may be, for example, a decrease in the severity of the symptoms associated with the mental, neurological or physiological condition; an eradication of the symptoms associated with the mental, neurological or physiological condition; or an increase in a mood that masks the symptoms associated with the mental, neurological or physiological condition.

The methods according to the present invention may be used therapeutically to address undesirable mental, neurological or physiological conditions in a patient. For example, the methods of the present invention may be used to treat a prrexisting undesirable mental, neurological or physiological condition in a patient, such as a mood disorder, an eating disorder, a pain disorder, a substance abuse disorder, an anxiety disorder, or an obsessive-compulsive disorder. The methods may also be used to reduce any future undesirable mental, neurological or physiological condition that is anticipated to occur, for example, due to future physical exertion, physical trauma, mental trauma, or medical procedure. Many examples of conditions that may be addressed using the methods of the present invention are described in more detail below.

### The Substance P System

According to one embodiment of the invention, the neurotransmitter system is the Substance P (“SP”) system, which includes as neurotransmitters the neurokinins Substance P, NKA and NKB. SP is a polypeptide and is known to act as a neurotransmitter and mediator for pain sensations. It is a member of the tachykinin family, which is a set of polypeptides having a similar C-terminal and a varying N-terminals with varying SP-like activity. The SP receptors include NK-1, NK-2 and NK-3 receptors. SP preferentially binds to NK-1 receptors, NKA preferentially binds to NK-2 receptors, and NKB preferentially binds to NK-3 receptors.

SP and its receptors are found primarily in the brain and spinal cord tissue. In the spinal cord, SP receptors are found in an area called the dorsal horn, which is a primary site for pain signals to be transmitted to the brain. In the brain, SP and its receptors are found in large concentrations in the hypothalamus and the amygdala, areas associated with affective behavior, anxiety and response to stress, and pain. In addition, SP is also implicated in nausea and emesis, defensive behavior, cardiovascular tone, salivary secretion, inflammation, smooth muscle contraction and vasodilation, as well as in numerous mental conditions such as schizophrenia, manic depressive psychosis, sexual dysfunction, drug addiction, cognitive disorders, locomotive disorders, and depression.

When the neurotransmitter system is the SP system, the type of receptor is SP receptors, which are positively linked to many undesirable mental and neurological conditions, and the ligand is an SP receptor agonist. The counteradaptation causes a down-regulation of the SP system, and may be at least one of a decrease in the biosynthesis or release of SP, NKA and/or NKB at the receptor terminals or by the pituitary gland; a decrease in the number of the receptors and/or binding sites on the receptors; or a decrease in the sensitivity of the receptors to binding by SP receptor agonists and/or SP, NKA and/or NKB.

The SP receptor agonist may be, for example, peptide-based. In certain embodiments of the invention, the SP receptor agonist is an analogue of SP, NKA, and/or NKB, or a pharmaceutically acceptable salt or derivative thereof. For example, the SP receptor agonist may be Substance P; Substance P, free acid; Biotin-Substance P; [Cys^{3,6}, Tyr⁸, Pro⁹]-Substance P; (Disulfide bridge: 3-6), [Cys^{3,6}, Tyr⁸, Pro¹⁰]-Substance P; (Disulfide bridge: 3-6), [4-Chloro-Phe^{7,8}]-Substance P; [4-Benzoyl-Phe⁸]-Substance P;

[Succinyl-Asp⁶, N-Me-Phe⁸]-Substance P (6-11)(Senktide); [Tyr⁸]-Substance P; [Tyr⁹]-Substance P; Shark Substance P Peptide; GR73632 [D-Ala-[L-Pro⁹,Me-Leu⁸]substance P(7-11)]; [Sar⁹,Met(O₂)¹¹]SP; GR 73,632 [delta-Aminovaleryl[Pro9, N-Me-Leu10]-substance P(7-11)], [Glu(OBzl)11] substance P and hemokinin 1 (HK-1) (a substance P homolog); or a pharmaceutically acceptable salt or carrier thereof.

In other embodiments of the invention, the SP receptor agonist may be an NKA or NKB analogue having a C-terminal heptapetpide similar to NKA(4-10) or NKB(4-10), or a pharmaceutically acceptable salt or carrier thereof. For example, the SP receptor agonist may be [Gln⁴]-NKA, [Gln⁴]-NKA(4-10), [Phe⁷]-NKA, [Phe⁷]-NKA(4-10), [Ile⁷]-NKA, [Ile7]-NKA(4-10), [Lys⁵,MeLeu⁹,Nle¹⁰]-NKA(4-10), [Nle¹⁰]-NKA(4-10), β-Ala⁸]-NKA(4-10), [Ala⁵]-NKA(4-10), *[Gln⁴]-NKB, [Gln⁴]-NKB(4-10), [Phe⁷]-NKB, [Phe⁷]-NKB(4-10), [Ile⁷]-NKB, [Ile7]-NKB(4-10), [Lys⁵,MeLeu⁹,Nle¹⁰]-NKB(4-10), [Nle¹⁰]-NKB(4-10), [β-Ala⁸]-NKB(4-10), [Ala⁵]-NKB(4-10), or a pharmaceutically acceptable salt or carrier thereof. Similarly, the SP receptor agonist may be [Arg]-NKB, an NKA or NKB analogue having Val⁷ replaced with MePhe, or a pharmaceutically accepted salt or carrier thereof.

Other SP receptor agonists that may be used in the present invention are SR 48968, an NK2 receptor antagonist ((S)—N-methyl-N[4-(4-acetylamino-4-[(phenyl piperidino)-2-(3,4-dichlorophenyl)-butyl]benzamide]) as well as those described in U.S. Pat. Nos. 4,839,465; 4,472,305; 5,137,873; 4,638,046; 4,680,283; 5,166,136; 5,410,019; and 6,642,233, each of which is incorporated herein by reference in its entirety.

The initial dosage (i.e., the dosage at the first administration) of the SP receptor agonist is desirably high enough to induce a counteradaptive effect, but not so high as to cause intolerable direct effects from ligand-receptor binding. For example, the initial dosage of the SP receptor agonist may be between about 0.5 pmol/kg/min and about 20 pmol/kg/min for continuous dosing during the first time period. In certain desirable embodiments of the invention, the initial dosage of the SP receptor agonist is between 3 pmol/kg/min and 10 pmol/kg/min for continuous dosing during the first time period.

The present invention is not limited to the use of peptide-based SP receptor agonists. Other SP receptor agonists, including substantially or wholly non-peptidic SP receptor agonists (e.g., those described in Chorev et al., Biopolymers, May 1991; 31(6):725-33), which is hereby incorporated herein by reference in its entirety) may be used in the methods of the present invention.

The SP receptor agonist may be administered using any appropriate route. Transmucosal administration is an especially desirable method for administering SP receptor agonists. For example, the administration may be sublingual or via rectal suppository. It may be desirable to administer the SP receptor agonist using both a rapidly absorbed loading dose (in order to get a fast binding of the SP receptors), and a gradually absorbed dose (in order to maintain a desired level of agonist-receptor binding over the desired length of the first time period). A rectal suppository having a rapidly-absorbing outer covering and a more slowly absorbing center could be used for such an administration. Alternatively, the loading dose could be given sublingually, and the gradually absorbed dose could be given transdermally via patch. Other routes include intraspinal or intrathecal administration for pain.

Desirably, an SP receptor antagonist is not administered during the first time period associated with each administration. In certain embodiments of the invention, however, an SP receptor antagonist is administered during one or more of the second time periods. Non-limiting examples of SP receptor antagonists along with suggested dosages are as follows: SR 48968 ((S)—N-methyl-N(4-acetylamino-4-phenylpiperidino-2-(3,4-dichloophenyl)butyl)benzamide); Osanetant and compounds described in U.S. Pat. Nos. 5,972,938; 6,576,638; 6,596,692; 6,509,014; 6,642,240; 6,841,551; 6,177,450; 6,518,295; U.S. Pat. No. 6,369,074; AND U.S. Pat. No. 6,586,432; AND WO 95/16679; 95/18124; 95/23798.

Other SP (NK₁) receptor antagonists include: L-760735 ([1-(5-{[(2R,3S)-2-({(1R)-3,5-bis(trifluoromethyl)phenyl]ethyl}oxy)-3-(4-phenyl)morpholin-4-yl]methyl}-2H-1,2,3-triazol-4-yl)-N,N-dimethylmethanamine]) (See Boyce, S, et al. Neurophannacology. 2001 July; 41(1):130-7); CP-96,345 [(2S,3S)-cis-2-(diphenylmethyl)-N-[(2-methoxy-phenyl)-methyl]-1-azabicyclo[2.2.2]-octan-3-amine] (See Snider, et al, Science, 1991 Jan. 25; 251(4992):435-7); SSR240600 ([(R)-2-(1-{2-[4-{2-[3,5-bis(trifluoromethyl)phenyl]acetyl}-2-(3,4-dichlorophenyl)-2-morpholinyl]ethyl}-4-piperidinyl)-2-methylpropanamide] (See Steinberg, R. et al., Steinberg, R, et al, J Pharm Exper Ther, 303(3), 1180-1188, December 2002, “SSR240600 [(R)-2-(1-{2-[4-{2-[3,5-Bis(trifluoromethyl)phenyl]acetyl}-2-(3,4-dichlorophenyl)-2-morpholinyl]ethyl}-4-piperidinyl)-2-methylpropanamide], a Centrally Active Nonpeptide Antagonist of the Tachykinin Neurokinin 1 Receptor: II. Neurochemical and Behavioral Characterization”); NKP608 [quinoline-4-carboxylic acid [trans-(2R,4S)-1-(3,5-bis-trifluoromethyl-benzoyl)-2-(4-chloro-benzyl)-piperidin-4-yl]-amide)] (see Spooren W P, et al., Eur J Pharmacol. 2002 Jan. 25; 435(2-3):161-70 and File, S E, Psychopharmacology (Berl). 2000 September; 152(1):105-9, entitled “NKP608, an NK1 receptor antagonist, has an anxiolytic action in the social interaction test in rats.”); L-AT (N-acetyl-L-tryptophan 3,5-bis benzyl ester) (See Crissman, A, et al., Vol. 302, Issue 2, 606-611, August 2002, entitled “Effects of Antidepressants in Rats Trained to Discriminate Centrally Administered Isoproterenol”); MK-869 [Aprepitant] (See Varty, G B, et al., Neuropsychopharmacology (2002) 27 371-379, “The Gerbil Elevated Plus-maze II: Anxiolytic-like Effects of Selective Neurokinin NK1 Receptor Antagonists”); L-742,694 [2(S)-((3,5-bis(Trifluoromethyl)benzyl)-oxy)-3(S)phenyl-4-((3-oxo-1,2,4-triazol-5-yl)methyl)morpholine] (See Varty, et al.); L-733060 [(2S,3S)3-([3,5-bis(trifluoromethyl)phenyl]methoxy)-2-phenylpiperidine] (See Varty, et al.); CP-99,994 [(+)-(2S,3S)-3-(2-methoxybenzylamino)-2-phenylpiperidine] (See McLean, et al, J Pharm Exp Ther, Volume 267, Issue 1, pp. 472-479 and Varty, et al.); CP-122,721 [(+)-(2S,3S)-3-(2-methoxy-5-trifluoromethoxybenzyl)amino-2-phenylpiperidine] (See McLean, et al., J Pharm Exp Ther, Volume 277, Issue 2, pp. 900-908 and Varty, et al); CP-96,345 [(2S,3S)-cis-2-(diphenylmethyl)-N-((2-methoxyphenyl)-methyl)-1-azabicyclo(2.2.2.)-octan-3-amine] (see Bang, et al., J Pharmacol Exp Ther. 2003 April; 305(1):31-9); GSK 597599 [Vestipitant]; GSK 679769 (See Hunter et al. U.S. Patent Publication no. 20050186245); GSK 823296 (See Hunter et al. U.S. Patent Publication no. 20050186245); Saredutant (See Van Schoor, et al., Eur Respir J 1998; 12: 17-23; Talnetant; Osanetant (see Kamali, F, Curr Opin Investig Drugs. 2001 July; 2(7):950-6); SR-489686 (benzamide, N-[4-[4-(acetylamino)-4-phenyl-1-piperidinyl]-2-(3,4-dichloro-phenyl)butyl]-N-methyl-(S)—); SB-223412 (See Hunter et al. U.S. Patent Publication no. 20050186245); SB-235375 (4-quinolinecarboxamide-,3-hydroxy-2-phenyl-N-[(1S)-1-phenylpropyl]-), UK-226471 (See Hunter et al. U.S: Patent Publication no. 20050186245).

Suitable but non-limiting initial dosages for SP receptor antagonists include about 12 mg/kg/hour/administration for 8 hours of L-760735 (via iv); about 30 μg/kg/hour/administration for 8 hours of CP-96,345 (via iv); about 0.1 and 10 mg/kg/administration of SSR240600 (via ip or po); about 0.01 to 0.1 mg/kg/administration of NKP608 (via po); about 1 to 10 mg/kg/administration of L-AT; about 0.01 to 3 mg/kg/administration of MK-869; about 1 to 30 mg/kg of L-742,694; about 1 to 10 mg/kg/administration of L-733,060; about 3 to 30 mg/kg/administration of CP-99,994 or CP-122,721; and about 100 mg/administration of Saredutant (via po).

The SP neurotransmitter system is positively linked to a wide variety of undesirable mental and neurological conditions. Examples of such conditions include chronic pain, mood disorders, eating disorders, anxiety disorders, motivational problems, substance abuse disorders, inflammatory conditions, nausea or emesis (e.g., arising from chemotherapy), urinary incontinence, skin rashes, erythema, eruptions, fibromyalgia, chronic fatigue syndrome, chronic back pain, chronic headaches, chronic cancer pain, shingles, reflex sympathetic dystrophy, neuropathy, inflammatory pain, pain that is anticipated to occur in the future (e.g., from a medical procedure or physical exertion), major depressive disorders, post-traumatic depression, temporary depressed mood, manic-depressive disorder, dysthymic disorder, generalized mood disorder, anhedonia, non-organic sexual dysfunction, overeating, obesity, anorexia, bulimia, generalized anxiety state, panic disorders, phobias, obsessive-compulsive disorder, attention deficit hyperactivity disorder, Tourette's Syndrome, hysteria sleep disorders, breathing-related sleep disorders, a lack of motivation due to learning or memory problems, abuse of substances such as narcotics, alcohol, nicotine, stimulants, anxiolytics, CNS depressants, hallucinogens and marijuana, asthma, arthritis, rhinitis, conjunctivitis, inflammatory bowel disease, inflammation of the skin or mucosa, acute pancreatitis. The down-regulation of the SP system desirably causes a therapeutic benefit with respect to the undesirable mental, neurological or physiological condition.

Virtually all types of pain, with the exception of acute sharp pain, are associated with the SP system. SP is not involved with the initial pain that is caused by a stabbing wound. The pain that lingers afterwards, however, is due to the SP pathway. In a similar manner the pain that lingers for a period of time after a surgical procedure is mediated by the SP pathway.

Mood is mediated through the SP system. Increased levels of SP are found in clinically depressed patients. Substance abusers have elevated levels of SP and, for those times when they are not on the abused substance, generally have a depressed and/or dysphoric mood. Clinical depression and substance abuse are thus both associated with an up regulation of the SP system. The pleasurable experiences of morphine are absent in mice that lack the SP receptor. Such mice do not become addicted to morphine (Murtra, et al., Nature 405, 180-183, May 11, 2000). Because opiates alone cannot induce euphoria, the Murtra study suggests that the SP system is the final pathway by which opiate euphoria is mediated. The fact that SP antagonists can acutely improve mood is consistent with this finding. Anxiety, response to stress, sexual dysfunction and eating disorders are largely related to mood, and are therefore also affected by the SP system.

The SP system has also been implicated in asthma (Kudlacz E. M., “Combined tachykinin receptor antagonists for the treatment of respiratory diseases”, Expert Opinion on Investigational Drugs, Vol. 7, No. 7, July 1998, pp. 1055-1062) nausea/emesis, cancerous tumor growth and metastasis (Palma, C, et al., Br. J. Cancer, 1999 January; Vol. 79(2): 236-43 and Friess, et al., Lab. Invest. 2003 May; Vol. 83(5):731-42), and urinary incontinence (Andersson K E, Experimental Physiology, Vol. 84(1), 195-213).

Methods of the present invention using SP receptor agonists as ligands may be used to address undesirable mental, neurological or physiological conditions in patients. For example the methods of the present embodiment of the invention may be used to address any of the above-listed conditions. The methods according to the present embodiment of the invention may also be used as an adjunct treatment for cancer (e.g., to decrease tumor growth and metastasis).

The methods of the present invention could also be used with an SP agonist in chronic recurring pain situations such as migraine headaches. Similarly, because the SP system is up-regulated in chronic pain syndromes, they may also be treated using the methods of the present invention with an SP agonist. Such chronic pain syndromes include pain due to nerve injury, neuropathies, chronic low back pain, reflex sympathetic dystrophy, cancer pain, shingles and arthritis.

The methods of the present invention can be used with SP agonists in the prophylaxis of pain prior to an event that is associated with pain. The methods of the present invention may be used in order to decrease post-operative pain and also to increase post-operative response to narcotic pain medications, which results in a lower dose of narcotics to obtain an analgesic effect. Similarly, an SP agonist could be used in the methods of the present invention prior to such pain-inducing competitive events such as football, hockey, and boxing. An SP agonist could be used prior to any competitive event, such as long distance running in order to reduce pain perceptions that are inevitable with such muscle and leg overuse activities. A reduced pain response ultimately allows the athlete to push him/her self to a greater extent, resulting in an improved performance.

The methods of the present invention may also be used with SP agonists in order to address anxiety, stress response, sexual dysfunction and eating disorders may be improved with the SP agonist CAT protocol. These conditions are largely related to mood, thus an improvement in conditions such as these are indirectly related to overall mood as opposed to a direct effect.

The methods of the present invention may also be used with SP agonists in order to address any or all addictive disorders. For example, the methods of the present invention can be used to address the abuse of substances such as narcotics, alcohol, nicotine/cigarettes, stimulants, anxiolytics, CNS depressants, hallucinogens and marijuana. Furthermore, gambling and electronic gaming addictions follow the same brain abnormalities as do substance abuse problems, and can also be addressed using the methods of the present invention.

The methods of the present invention may also be used with SP agonists in order to address asthma by decreasing the severity of asthma attacks. An inhalational route of administration may be used in order to concentrate the counteradaptive effect in the lungs where it is most needed. The methods of the present invention may also be used with SP agonists in order to decrease the inflammatory response in any one of a number of inflammatory conditions such as arthritis, rhinitis, conjunctivitis, inflammatory bowel disease, inflammation of the skin and mucosa and acute pancreatitis. The methods of the present invention may also be used with SP agonists in order to address nausea/emesis, especially that associated with chemotherapy for cancer, and urinary incontinence.

### The Endogenous Endorphin System

According to another embodiment of the invention, the neurotransmitter system is the endogenous endorphin system, which includes as neurotransmitters the endorphins that bind preferentially to mu and/or delta opiate receptors. Endorphins are endogenous opiate-like compounds that act through their effects on the binding of opiate receptors. Mu and delta opiate receptors act in unison, and are stimulated by opiate and opiate-like compounds. Mu receptors primarily modulate pain, but also modulate mood. Delta receptors have the opposite focus, primarily modulating mood, but also modulating pain.

When the neurotransmitter is the endogenous endorphin system, the type of receptor is mu and/or delta opiate receptors, which are generally negatively linked to undesirable mental and neurological conditions. Mu opiate receptors are associated primarily with lower levels of pain when stimulated, while delta opiate receptors are associated primarily with euphoria when stimulated. The ligand is a mu and/or delta opiate receptor antagonist, and the counteradaptation causes an up-regulation of the endogenous endorphin system. The counteradaptation may be, for example, an increase in the biosynthesis or release of endorphins at receptor terminals and/or by the pituitary gland; an increase in the number of the receptors and/or endorphin binding sites on the receptors; an increase in the sensitivity of the receptors to binding by mu and/or delta receptor agonists and/or endorphins; or any combination thereof.

The method according to the present embodiment of the invention may be practiced using a specific mu receptor antagonist or a specific delta receptor antagonist. For example, the method may be practiced using a specific mu receptor antagonist such as clocinnamox mesylate, CTAP, CTOP, etonitazenyl isothiocyanate, β-funaltrexamine hydrochloride, naloxonazine dihydrochloride, Cyprodime, and pharmaceutically acceptable salts, analogues, and derivatives thereof. The method may also be practiced using specific delta receptor antagonists such as naltrindole, N-benzylnaltrindole HCl, BNTX maleate, BNTX, ICI-154,129, ICI-174,864 (N,N-diallyl-Tyr-Aib-Aib-Phe-Leu-OH, where Aib is alpha-amino-isobutyric acid), naltriben mesylate, SDM25N HCl, 7-benzylidenenaltrexone, and pharmaceutically acceptable salts, analogues, and derivatives thereof. The skilled artisan may also employ non-specific mu and/or opiate antagonists, such as naloxone and naltrexone, in the methods according to the present embodiment of the invention. Non-limiting representative examples of non-specific opiate antagonists include Nalorphine, nalbuphine, levallorphin, cyclazocine, diprenorphine

Other mu and/or delta opiate receptor antagonists useable in the methods of the present invention include those described in U.S. Pat. Nos. 5,922,887; 4,518,711; 5,332,818; 6,790,854; 6,770,654; 6,696,457; 6,552,036; 6,514,975; 6,436,959; 6,306,876; 6,271,239; 6,262,104; 5,552,404; 5,574,159; 5,658,908; 5,681,830; 5,464,841; 5,631,263; 5,602.099; 5,411,965; 5,352,680; 5,332,818; 4,910,152; 4,816,586; 4,518,711; 5,872,097; 5,821,219; 5,326,751; 4,421,744; 4,464,358; 4,474,767; 4,476,117; 4,468,383; 6,825,205; 6,455,536; 6,740,659; 6,713,488; 6,838,580; 6,337,319; 5,965,701; 6,303,578; and 4,684,620, and International Patent Application WO/2004/026819 each of which is incorporated herein by reference in its entirety.

In certain desirable embodiments of the invention, the mu and/or delta opiate receptor antagonist is naloxone, naltrexone, nalmefene, or nalbuphine, or a pharmaceutically acceptable salt or derivative thereof. Naltrexone is a desirable mu and/or delta receptor antagonist, but may not be usable in all situations due to its long compound half-life (48-72 hours); while naltrexone itself has a half-life of 9-10 hours, its active metabolites (e.g. 6-beta-naltrexol and 2-hydroxy-3-methoxynaltrexol) have much longer half-lives. Naloxone is an especially desirable mu and/or delta receptor antagonist for use in the present embodiment of the invention. Naloxone has a compound half-life of about an hour, but cannot be given orally. Naloxone can be given intravenously or through a transdermal patch, desirably using a time-release formulation. Suitable transdermal patches are described in U.S. Pat. No. 4,573,995, which is hereby incorporated herein by reference in its entirety.

Naloxone has a half-life of 1-1.5 hours, which is favorable for use in the methods of the present invention. Although the 1-1.5 hour half-life is adequate for inducing a counteradaptive response, amore preferred ligand for use in the present invention would have a half-life between 2-4 hours. Furthermore, naloxone has very poor oral bioavailability, being only 5%, making it less convenient for outpatient administration.

Thus in one aspect, the present invention provides methods using 3-hydroxymorphinans and derivatives and prodrugs thereof as mu and/or delta opiate antagonist ligands that are orally bioavailable, and that have a half-life that is longer than that of native naloxone, yet less than the 8 hour half-life of naltrexone or nalmefene. The preferable compounds have half-lives that are 4 hours or less, and are desirably in the range of 2-4 hours. Optionally, the 3-hydroxymorphinans as mu and/or delta opiate antagonist ligands may be given by a transmucosal route.

The bioavailablity of 3-hydroxymorphinan compounds may be increased through the use of prodrug formulations. The pro-drug formulations for the invention herein involve the attachment of a chemical group(s) to the native 3-hydroxymorphinan compound in order to prevent the first pass metabolism process, which is the rapid glucuronidation of the 3-OH moiety. The attached chemical groups for the invention herein are non-toxic. Furthermore, the attached chemical groups are those that are removed in a period of time such that the overall compound half-life of the mu and/or delta opiate antagonist ligand remains less than 4 hours.

Some of the 3-hydroxymorphinan compounds described herein for use in the present invention have enhanced lipophilicity. Greater lipophilicity enhances transmembrane absorption, which acts to improve oral and transmucosal bioavailability. It further results in a more rapid and efficient transport across the blood-brain barrier. Such improved transport across the blood-brain barrier is preferable in that it results in a greater counteradaptive response by the mu and/or delta opiate receptor antagonist.

In desirable embodiments of the invention, the mu and/or delta opiate receptor antagonist is a naloxone analog, due to the fact that naloxone analogs have a relatively shorter half-life than do other opiate antagonists, such as naltrexone or nalmefene.

In order to make naloxone orally, and/or transmucosally, bioavailable for outpatient (or inpatient) counteradaptive therapy purposes, numerous naloxone pro-drug chemical formulations are desirably used. These pro-drugs include modifications at the 3-OH moiety, the 6-Carbon, the C-14 Carbon and N-oxide pro-drug formulations. The preferred modifications are at the 3-OH and/or the 6-Carbon sites.

### 3-OH Modifications

Morphine Example:

Morphine analogs are used in order to demonstrate modifications that result in a more lipophilic structure and how such modifications are beneficial. The morphine structure is:

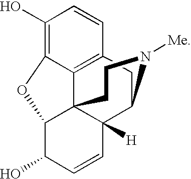

When the 3-OH and 6-OH groups are converted to acetyl groups, the compound becomes diacetylmorphine, which is otherwise known as heroin. Diacetylmorphine has properties that are favorable to morphine in that it is more lipophilic, therefore it is more rapidly and efficiently absorbed across mucosal surfaces. For example, oral bioavailability of morphine is @ 25-30% (Hasselstrom, J, et al., Clin Pharmacokinet. 1993 April; 24(4): 344-54 and Gourlay, G K, et al., Pain. 1986 June; 25(3): 297-312.), whereas oral bioavailbility of diacetylmorphine is @ 67% (Girardin, F, et al., “Pharmiacokinetics of high doses of intramuscular and oral heroin in narcotic addicts.” Clin Pharmacol Ther. 2003 October; 74(4): 341-52.) Furthermore, the diacetyl derivative more readily crosses the blood-brain barrier.

For the naloxone compound a similar lipophilicity occurs when the 3-OH group is converted to the 3-acetylnaloxone derivative. This enhanced lipophilicity results in significant benefits over the native naloxone compound, including an improved bioavailability, enhanced potency and increased duration of action for 3-acetyl naloxone. These benefits are demonstrated by Linder and Fishman (“Narcotic Antagonists. 1 . . . .” J Medicinal Chemistry, 1973, 16(5): 553).

When the carbon chain is further lengthened, such as converting the 3-OH to 3-propanoyl, 3-butanoyl, 3-hexanoyl then the lipophilicity is further increased. For example, with respect to the 3,5 diesters of morphine, the addition of such lengthened carbon chain groups results in either similar or greater potencies, along with a longer duration of action, from 20%, up to 5 times as long. [Owen, J A, et al., “Morphine Diesters. I . . .” Can J Physiol Pharmacol. 1984 April; 62(4): 446-51 and “Morphine Diesters. II . . .” pgs 452-456.]

Desirable compounds for use in the present invention include similar 3-OH acetyl, butanoyl, propanoyl, hexanoyl derivatives of naloxone in order to improve lipophilicity (which acts to enhance the counteradaptive response), to improve oral bioavailability, and to slightly increase the duration of action of the naloxone compound.

Naltrexone Example:

Naltrexone is similar to the naloxone structure, the only difference being the N moiety. These differences are noted in the following sketches:

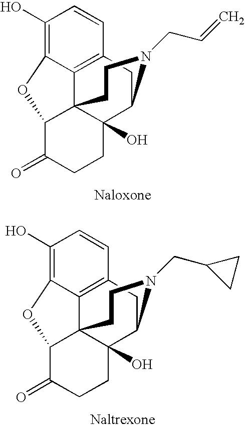

Enhanced oral bioavailability of 3-hydroxymorphinans can be demonstrated by analyzing 3-OH modifications of the compound naltrexone. Specifically, 3-OH modifications of the naltrexone compound, which consist of a benzoate analog ester, demonstrate enhanced bioavailability. For example, bioavailability is 28 to 45 times greater for the acetylsalicylate and anthranilate esters of naltrexone, respectively, as compared to the native naltrexone compound. (see Hussain, M A, et al., J Pharm Sci. 1987 May; 76(5): 356-8; Hussain M A, et al., Pharm Res. 1988 Feb. 5(2): 113-5; U.S. Pat. No. 4,668,685 and U.S. Pat. No. 4,673,679) The metabolic byproducts of these prodrugs of naltrexone are anthranilic acid and acetylsalicylate. Anthranilic acid is a normal-occurring metabolic byproduct of the amino acid tryptophan. Acetylsalicylic acid is better known as aspirin. Both of these byproducts are considered safe and non-toxic at the doses that would be required to induce counteradaptations.

Accordingly, in desirable embodiments of the invention, such anthranilic acid acetylsalicylic acid 3-OH prodrug modified naloxone compounds are used in the methods described herein. Furthermore, based on the increased lipophilicity and improved oral bioavailability of the carbonyl attachments at the 3-OH site, in other desirable embodiments of the invention alkanoyl (2-6 carbon atoms) prodrug modified naloxone compounds are used in the methods described herein.

### Carbon-6 Modifications

In certain desirable embodiments of the invention, the methods described herein are performed using naloxone derivatives modified at the 6-carbon. One such modification involves the conversion of the native 6-=O compound to its 6-desoxy, 6-methylene derivative (6-=CH₂), as described, for example, in U.S. Pat. No. 3,814,768 and U.S. Pat. No. 4,535,157.

The 6-methylene modification is a preferred one because such a conversion results in several benefits. These include enhancing the effects at the opiate receptor, improving oral bioavailability and making the molecule more lipophilic. These benefits are demonstrated when the 6-=CH₂ substitution is made to the naltrexone molecule, which results in the compound that is called nalmefene. The 6-methylene naltrexone analogue, nalmefene, has improved properites over naltrexone for several reasons, including: 1) greater bioavailability, 2) longer duration of antagonist action, 3) more competitive binding to opioid receptor [which would generate a greater counteradaptive response], and 4) no dose dependent liver toxicity. [Mason, B J, et al., Arch Gen Psych 1999, August; 56(8):719-24 and Dixon, R, et al., J Clin Pharm 1987; 27:233-239.]

A further example of improved receptor activity with the 6-=CH₂ substitution is demonstrated by the 6-methylene derivatives of morphine. Such analogs are 75 times more potent than the parent morphine compound (see Hahn and Fishman, J Med Chem. 1975, 18(3): 259.). Hahn and Fishman further indicate that the 6-methylene naloxone derivative has considerably superior oral potency as compared to the native naloxone compound.

Another such modification involves the conversion of the native 6-=O compound to its 6-desoxy (ie., 6-(—H)₂) analog. The 6-desoxy modification has several potential benefits over the 6-=O group. First, it makes the compound more lipophilic. Second, it increases opiate receptor activity. For example, 6-desoxymorphine has 10 times the activity as does the native morphine compound. Similarily, 6-desoxy naloxone has enhanced antagonist activity as compared to the native naloxone compound (see Table 1, Materials & Methods, Minakami, et al., Life Sciences 1962, 10; 503-507.). Third, it is intended to increase the duration of activity, ie., increase the half-life, to a slight extent.

Other such modifications involve the conversion of the native 6-=O compound to 6-OH, 6-amine, or 6-amide analogs. U.S. Pat. No. 6,713,488 describes ‘Neutral antagonists’ where modifications are made at the 6-Carbon group. These compounds are said to be ‘neutral’ because the 6 Carbon ketone group is converted to an —OH functionality, or an amine, or amide or the like.

Simultaneous modifications may be made at both, the 3-OH and 6-C═O sites. Such double modifications are intended to even further enhance oral bioavailability and the counteradaptive response. For example, because an anthranilic acid 3-OH substitution enhances oral bioavaiability 45 times, and because converting 6-C═O group into either a 6-methylene or 6-desoxy moiety also enhances oral bioavailability, the two (3-OH and 6C modifications) together are expected to enhance oral availability even more. Moreover, the two substitutions further act to improve transport across the blood-brain barrier and enhance opiate receptor activity, which both act to enhance the counteradaptive response, thus improving clinical efficacy.

### Other Modifications

N-oxide derivatives of 3-hydroxymorphinans may also be used in the methods of the present invention. Such derivatives are described in U.S. Pat. Nos. 4,722,928 and 4,990,617.

Derivatives of hydroxymorphinans having substitution at the 14-Carbon may also be used in the methods of the present invention. U.S. Pat. No. 4,912,114 and U.S. Pat. No. 4,272,540, which are incorporated herein by reference, describe 3-hydroxymorphinan compounds with substitutions at the 14-Carbon group.

Accordingly, in certain embodiments of the invention, the mu and/or delta opiate receptor antagonist is a 3-morphinan compound having the structure

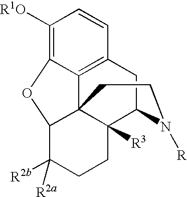

wherein R is allyl, methylallyl, cyclopropylmethyl, dimethylallyl, tetrahodrofurfuryl or cyclobutylmethyl; R¹ is H, (C₁-C₁₈ hydrocarbyl)-, (C₁-C₁₈ hydrocarbyl)-CO—, (C₁-C₁₈ hydrocarbyl)₂N—CO—, (C₁-C₁₈ hydrocarbyl)-SO₂—, (C₁-C₁₈ hydrocarbyl)-O—CO—, Ph-CO—, Ph-SO₂—, Ph-NH—CO, wherein each Ph is optionally substituted with one or more substituents independently selected from the group consisting of (C₁-C₁₂ hydrocarbyl), (C₁-C₁₂ hydrocarbyl)-O—, Cl, F, Br, I, CF₃, R⁴O—, and R⁴₂N—, in which each R⁴ is independently selected from the group consisting of H, (C₁-C₄ alkyl), H—CO— and (C₁-C₄ alkyl)-CO—; each R^{2a} and R^{2b} is independently selected from the group consisting of H, (C1-C6 alkyl), (C₁-C₆ alkyl)-O—, (C₁-C₆ alkyl)-CO—O—, R⁵—O—, R⁵₂N—, R⁵—CO—NH—, R⁵—S—, and NO₂, in which each R⁵ is independently selected from the group consisting of H, (C₁-C₆ (C₃-C₁₀ cycloalkyl), (C₆-C₁₀ aryl), (C₁-C₆ alkyl)-CO—, (C₃-C₁₀ cycloalkyl)-CO—, (C₆-C₁₀ aryl)-CO—, each of which is optionally substituted with 1-3 substituents selected from the group consisting of (C₁-C₁₂ hydrocarbyl), (C₁-C₁₂ hydrocarbyl)-O—, Cl, F, Br, I, CF₃, R⁴O—, and R⁴₂N—; or R^{2a} and R^{2b} together form O═ or CH₂═; and R³ is H, OH, CH₃ or OCH₃, or an N-oxide or pharmaceutically acceptable salt thereof. These compounds are described in more detail in U.S. Provisional Patent Application Ser. No. 60/858,186, entitled “OPIATE ANATOGONISTS FOR COUNTERADAPTATION THERAPY,” which is incorporated herein by reference.

The compounds having the above-described structure may be desirable as they can be formulated to have a compound half life of 2-4 hours, longer than that of naloxone, but shorter than that of naltrexone. Further, substitution at the 3-OH (i.e., compounds with R¹ not H) can give the compounds greater oral bioavailablity than naloxone due to blocking of metabolic reactions at the 3-OH. Accordingly, these compounds may be formulated in more convenient oral administration forms. The R¹ moiety is preferably non-toxic when it is cleaved from the 3-OH to form its corresponding alcohol, acid or amide. The compounds may also be formulated to have greater lipophilicity, which can enhance transmembrane absorption, which can improve oral and transmucosal bioavailability as well as increase transport across the blood-brain barrier.

Synthetic methods to make compounds having the above-described structure may be derived by the skilled artisan from methods known in the art, such as those described in U.S. Pat. Nos. 5,366,979; 6,713,488; 6,784,187; 4,912,114; 4,272,540; 4,322,426; 4,722,928; 4,990,617; 4,673,679; 4,668,685; 6,569,449; 4,535,157 and 5,908,846, as well as in U.S. Provisional Patent Application 60/813,845, filed Jun. 15, 2006 and entitled “ORALLY AVAILABLE NALOXONE DERIVATIVES AND METHODS OF SYNTHESIS,” each of which is hereby incorporated herein by reference.

In certain embodiments of the invention, R is allyl. Compounds in which R is allyl tend to have desirably short compound half lives.

In certain embodiments of the invention, R¹ is not H. For example, R¹ may be o-aminobenzoyl or o-(acetyloxy)benzoyl. R¹ may also be (C₁-C₁₈ alkyl) or (C₁-C₁₈ alkyl)CO—. One especially desirable identity for R¹ is (C¹-C⁵ alkyl)CO—. As used herein, a (Cₙ-Cₘ alkyl) group is a straight-chain or branched alkyl chain having n-m carbon atoms.

In certain embodiments of the invention, R^{2a} and R^{2b} taken together do not make O═. Such compounds are more lipophilic and have greater opiate receptor activity than do the naloxone compound. For example, R^{2a} and R^{2b} may each be H, or may be taken together to form CH₂═.

In certain especially desirable embodiments of the invention, R¹ is not H and R^{2a} and R^{2b} taken together do not make O═. Such compounds have especially increased oral bioavailability and blood-brain barrier transport.

In certain embodiments of the invention, R³ is not OH.

One example of a suitable delta receptor selective antagonist has the structure:

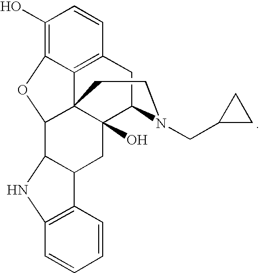

The initial dosage of the mu/and or delta opiate receptor is desirably high enough to induce a counteradaptive effect, but not so high as to cause the patient intolerable direct effects. For example, the initial dosage of the mu and/or delta opiate receptor antagonist may be equivalent to between about 2 mg/administration and about 200 mg/administration of naloxone. In certain desirable embodiments of the invention, the initial dosage of the mu and/or delta opiate receptor antagonist is equivalent to between about 10 mg/administration and about 100 mg/administration of naloxone.

When using naloxone as the mu and/or delta opiate receptor antagonist, the initial dosage may be between 5 and 500 mg/administration. Desirably, the initial dosage is between 10 and 50 mg/administration. In certain embodiments of the invention, each dosage of naloxone is greater than 10 mg/administration; greater than 10.5 mg/administration; greater than 11 mg/administration; or greater than 15 mg/administration. Desirably, the initial dose of naloxone is at least about 30 mg/administration (over 8 hour period), as this amount results in a complete blockade of opiate receptors. Desirably, the maximum dosage of naloxone is no greater than 3000 mg/administration.

In one example of a daily dosing regimen for naloxone, the initial dosage of naloxone is 30 mg/administration over an 8 hour period. After two weeks, the dosage is doubled. After another two weeks, the dosage is increased to 120-160 mg/administration. After another month, the dosage is increased to 300 mg/administration, then to 500-600 mg/administration after another two months. After another two months, the dosage is increased to 1000 mg/administration, then to 1500-2000 mg/administration after another two months. Alternatively, a much larger initial dose (e.g., 100-500 mg/administration) could be used in order to build up a counteradaptation more quickly. A low dose of naltrexone (e.g., 10-25 mg/administration) could be used along with the naloxone to realize an additional counteradaptive effect.

In one example of a dosing regimen for naltrexone, an initial dosage of 10-25 mg naltrexone is given daily. Alternatively, larger doses (e.g., 25-200 mg/administration) are given once, twice, or thrice weekly. With larger doses of naltrexone, the first time period will be relatively long, and may occasionally overlap with the waking hours of the patient.

The mu and/or delta opiate receptor antagonist may be administered orally, transdermally, intraspinally, intrathecally, via inhalation, subcutaneously, intravenously, intramuscularly, or transmucosally, or via osmotic pump, microcapsule, implant, or suspension. In certain embodiments of the invention (e.g., where the mu and/or delta opiate receptor antagonist has a relatively short compound half-life), it may be desirable to administer it using a time-release or slow-release formation, or transdermally (e.g., using a patch) in order to provide an administration half-life of sufficient length. When the mu and/or delta opiate receptor antagonist is administered transdermally or using a time-release or slow-release formulation, it is desirably released over a time period between two and twelve hours in duration; between two and six hours in duration; or between six and twelve hours in duration. In order to provide a high in vivo concentration of the ligand in a short amount of time, it may be desirable to administer the mu and/or delta opiate receptor antagonist using a rapidly absorbed loading dose. To provide a high in vivo concentration of the ligand quickly as well as a desirably long administration half-life, it may be desirable to use both a rapidly absorbed loading dose and transdermal administration or a time-release or slow-release formulation. A transdermal patch for naloxone, naltrexone and nalbuphine is disclosed in U.S. Pat. No. 4,573,995, which is hereby incorporated herein by reference in its entirety. The invention herein further includes the simultaneous use of oral and transmucosal opiate antagonist routes of administration. The reason for this is that the transmucosal application is intended to result in rather immediate high circulation levels of the opiate antagonist compound. When using one of the aforementioned prodrugs, such administration would also result in immediate high levels of intracranial opiate antagonist. Such rapid high levels of the opiate antagonist are important when the intent is to induce an optimal counteradaptive response.

Another aspect of the invention relates to the option that an oral dose of the opiate antagonist may be given along with the transmucosal dose. Whereas the transmucosal administration results in rapid circulating levels that last a short period of time, the oral compound has a gradual onset of circulating levels, and thus this form of administration is intended to maintain relatively high circulating levels of the opiate antagonist compound in circulation for a more prolonged period of time, ie., up to 8 hours. For example, if the native naloxone molecule or a 3-OH acetyl modification were administered by a transmucosal route, then the effects would only last in the circulation for 2-4 hours, which is in essence the result of a half-life of 1-1.5 hours for these compounds. Because the counteradaptive response will be maximized if the opiate antagonist effect is more prolonged, ie., up to 8 hours—the time spent sleeping, then the oral naloxone analog would result in a more prolonged period of time with elevated circulation levels of the naloxone. In other words, the dose that is given by the transmucosal route is intended to generate rapid circulation levels that last for a short period of time, while the oral dose would maintain the circulating levels for up to 8 hours.

Another aspect of the invention relates to the use of variable administration routes. For example, one may choose to begin therapy with one of the aforementioned 3-hydroxymorphinan compounds by a transmucosal route of administration. This would be done with lower doses of the compound in order to decrease acute side effects. After a period of time one may switch to oral administration, where larger doses would be more practical, and by which time the individual would have adapted to side effects.

In certain embodiments of the invention, it may be desirable to administer both a specific mu and/or delta receptor antagonist and a non-specific mu and/or delta opiate receptor antagonist. The two types of antagonist may be administered substantially simultaneously or sequentially. Because the non-specific antagonists generally provide a greater counteradaptive effect than do specific mu or delta opiate antagonists, it is desirable to administer non-specific antagonists in the early stages of the method.

Because the body develops a tolerance to anti-opiates about eight days after first administration, it may be desirable to increase the dosage of the mu and/or delta opiate receptor antagonist with time. For example, it may be desirable to increase the dosage with a period of between a week and two weeks.

Desirably, an endorphin receptor agonist is not administered during the first time period associated with each administration. In certain embodiments of the invention, however, an endorphin receptor agonist is administered during one or more of the second time periods. Suitable but non-limiting examples of endorphin agonists include opiates such morphine, codeine, hydrocodone, fentanyl, and oxycodone. Morphine may be administered at dosages of 1-20-50 mg i.v. or 1-50 mg/hour continuous release via any suitable means such as transdermal, SQ, IM, or pump; Fentanyl may be administered at dosages of 0.1-0.5 mg gradual release over 8 hours via any suitable means such as transdermal, SQ, IM, or pump; Codeine may be administered at dosages of 10-100 mg p.o. every 4-6 hours; Hydrocodone may be administered at dosages of 5-25 mg p.o. every 4-6 hours; Oxycodone may be administered at dosages of 5-100 mg p.o. every 4 hours by any suitable means such as slow release transdermal, i.m., or SQ over 4-8 hours).

Enkephalins having an amino acid sequence of H-Tyr-Gly-Gly-Phe-Met-OH or H-Tyr-Gly-Gly-PheLeu-OH or any active analogues of these amino acid sequences with pharmacologically accepted carriers. Enkephalins may be administered at dosages of 1.0 μg/hr continuous release (transdermal, i.v., SQ, i.p. i.m. infusion pump).

Beta endorphin (a 31 amino acid peptide) or any and all active analogues, eg., beta-endorphin-(1-26), [D-Ala2]beta-endorphin or [Leu5]beta-endorphin with accepted pharmacologically accepted carriers. Beta endorphins may be administered at dosages of 1.0 μg/hr continuous release (e.g. transdermal, iv., SQ, i.p. i.m. infusion pump).

Mu selective agonists such as Carfentanil which may be administered at a dosage of 1-25 μg/kg; [D-Ala2, NMe-Phe4, Gly-o15] enkephalin and any active analogue with pharmacologically accepted carriers. The enkephalins may be administered at a suggested dosage of 1.0 μg/hr continuous release (e.g. i.v., i.m., SQ, pump, or transdermal).

Delta selective agonists such as DPDPE ([D-Pen2,D-Pen5]enkephalin); SB-235863; and SNC 80. DPDPE may be administered at a suggested dosage of 1.0-5.0 μg/hr continuous release (e.g., i.v., i.m., SQ, pump, or transdermal). SB-235863, ([8R-(4bS*,8aα,8aβ,12bβ)]7,10-Dimethyl-1-methoxy-11-(2-methylpropyl)oxycarbonyl 5,6,7,8,12,12b-hexahydro-(9H)-4,8-methanobenzofuro[3,2-e]pyrrolo[2,3-g]isoquinoline Hydrochloride) may be administered at a dosage of 70 mg/kg p.o. See Paola Petrillo, et al. J. Pharmacology and Experimental Therapeutics, First published on Oct. 9, 2003; DOI: 10.1124/jpet.103.055590. SNC 80 may be administered at a dosage of 50-75 mg/kg slow release over several hours, transdermal, i.p. SQ, pump, etc.) See E J Bilsky, et al., Pharmacology and Experimental Therapeutics, Volume 273, Issue 1, pp. 359-366, Apr. 1, 1995.

It may be desirable to administer a CRF receptor antagonist during the second time period. Suitable CRF receptor antagonists include R121919, DMP696, antalarmin, CP-154,526, SSR125543A, 2-arylamino-4-trifluoromethyl-aminomethylthiazole antagonists, astressin, alpha-helical CRF compounds, as well as the compounds described in U.S. Pat. Nos. 5,132,111; 5,278,146; 5,824,771; 5,844,074; 6,214,797; 6,670,371; 6,812,210 and 6,953,838 (each of which is hereby incorporated herein by reference) and pharmaceutically acceptable salts, analogues, and derivatives thereof. The CRF system is described in more detail below.

It may be desirable to administer a CRF agonist in combination with the mu and/or delta opiate receptor antagonist, each administration of the CRF receptor agonist having an administration half-life, wherein the ratio of the administration half-life to the period between administrations is no greater than ½. Suitable CRF receptor agonists include analogues of corticotropin releasing factor, and pharmaceutically-accepted salts and derivatives thereof.

When using a CRF receptor agonist and/or an AVP receptor agonist in a conventional dosing regimen, it may be desirable to repeatedly administer a mu and/or delta opiate receptor antagonist as described herein. This may be desirable due to the fact that the use of a CRF receptor agonist and/or an AVP receptor agonist may have the unintended consequence of down regulating the release of beta endorphin from the anterior pituitary gland. Such a reduced endorphin release would induce the opposite effect as to what is needed for the improvement of undesirable conditions that correlate inversely with the levels of circulating endorphins, such as depression and anxiety disorders. The repeated administration of the opiate antagonist may have two important effects: to block the endorphin down regulation by the CRF receptor agonist and or the AVP receptor agonist, and also to cause an up-regulation of the endorphin system as described above. Accordingly, in some embodiments of the invention a CRF receptor agonist and/or an AVP receptor agonist is administered during the second time period associated with each administration of the mu and/or delta opiate receptor antagonist.

The endogenous endorphin system and its mu and/or delta opiate receptors are negatively linked to a wide variety of undesirable mental and neurological conditions. Examples of such conditions include pain, mood disorders, eating disorders, anxiety disorders, motivational problems, substance abuse disorders, insufficient motivation or performance, immune system-related conditions, wounds in need of healing, pain that is expected to occur in the future (e.g., due to a future operation or due to future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, a generalized anxiety state, panic disorders, Tourette's Syndrome, hysteria sleep disorders, breathing-related sleep disorders, lack of motivation due to learning or memory problems, abuse of a substance such as narcotics, alcohol, nicotine, stimulants, anxiolytics, CNS depressants, hallucinogens and marijuana, insufficient motivation or preparation for a desired mental or physical activity (e.g. physical training, athletics, studying or testing), immune-related condition such as infection, AIDS, or cancer, and wounds in need of healing. The up-regulation of the endogenous endorphin system desirably causes a therapeutic benefit with respect to the undesirable mental, neurological or physiological condition.

The endogenous endorphin system is implicated in pain because endorphins can bind to pain-mediating opiate receptors and decrease the synthesis of SP, a pain-inducing substance. The endogenous endorphin system has also been implicated in stress (U.S. Pat. Nos. 5,922,361 and 5,175,144), wound healing (Vinogradov V A, Spevak S E, et al, Bi and U.S. Pat. No. 5,395,398), substance abuse, eating disorders (Full & fulfilled: the science of eating to your soul's satisfaction, by Nan Allison; Carol Beck, Publisher: Nashville, Tenn.: A & B Books, ©1998, ISBN: 0965911799), motivational problems (Tejedor-Real, P, et al, Eur J Pharmacol. 1998 Jul. 31; 354(1):1-7); immune response (Wybran, Fed Proc. 1985 January; 44(1 Pt 1):92-4, and U.S. Pat. No. 5,817,628) and cancer (Zagon, I S, et al., Cancer Lett, 1997; 112:167-175; U.S. Pat. Nos. 6,737,397; 6,136,780; and 4,801,614).

The endogenous endorphin system is also implicated in mood. Euphoria is the most recognizable emotional effect of opioids, which gives one an elevated feeling of well-being and care-free. Euphoria is modulated by endogenous endorphins. Endorphins are released with pleasurable experiences such as eating, exercise, winning an event, romantic encounters. It is thought that the endorphin release generates a feeling of well-being as a ‘reward’, which acts as a motivational mechanism in order to inspire an individual to fulfill nutritional and reproductive requirements. Another function of the endogenous endorphin system with respect to mood is to decrease anxiety, especially with regards to stress response. Rang H. P. (1995). Peptides as Mediators. In H. P. Rang & M. M. Dale, Pharmacology, Churchill Livingstone, N.Y.) demonstrates that endorphins are released at times of emotional stress, which acts to induce euphoria in order that anxiety is reduced.

Both endogenous endorphins and synthetic opiates may induce euphoria. The difference is that endogenous endorphins are rapidly degraded at their synapse and receptor sites, such that the effect is short term. With a short term effect there is no development of tolerance or dependency. Synthetic opiates, such as narcotics, have a much longer reactive time, thus they are associated with the development of dependency. Synthetic opiates have not been developed that have both, a strong analgesic effect and little or no potential for the development of dependency. Because endogenous endorphins have a similar euphoria-inducing capability as do opiates it is advantageous to use endogenous endorphins for inducing an elevated mood. However, because the administration of relatively large and prolonged doses of synthetic endorphins may be associated with the development of tolerance and dependency, they are not desirable long-term treatment agents.

Both mu and delta opiate receptors are involved to some degree with mood. Mu receptors primarily mediate pain perception, but also induce euphoria when these receptors are bound by endorphin/opiate compounds. The role of delta receptors in pain modulation is not clear, whereas they are likely more closely related to euphoria. Delta receptor agonists demonstrate anti-depressant activity in rats in the forced swim assay. Furthermore, evidence from animal studies demonstrates that delta-opioid receptors are involved in motivational activities. Their preferential involvement is through enkephalin-controlled behavior. Broom, et al. (Jpn J. Pharmacol. 2002 September; 90(1):1-6) demonstrate that the delta opiate receptor plays a significant role in depression.

Methods of the present invention using mu and/or delta receptor antagonists as ligands may be used to address undesirable mental, neurological or physiological conditions in patients. For example the methods of the present embodiment of the invention may be used to address any of the above-listed conditions. The methods according to the present embodiment of the invention may also be used as an adjunct treatment for cancer.

Methods of the present invention using mu and/or delta receptor antagonists may be used to address pain that is anticipated to occur in the future. For example, if a patient is scheduled for elective surgery in, e.g., one month then the method of the present invention can be practiced with a mu and/or delta opiate receptor, using high night-time dosing for the intervening pre-operative period of time. After surgery the patient will have an enhanced response to pain due to the up-regulated endogenous endorphin system. In addition, the patient will require lower overall doses of narcotic pain medications post-operatively due to enhanced sensitivity of mu and/or delta opiate receptors. The method would likely best interrupted immediately after surgery so that post-operative pain would not increase due to the direct effects of receptor antagonism. It could be restarted in a few days or so, once the pain had subsided, in order to maintain the counteradaptive response.

In an example of a pre-operative treatment according to the present invention, a 49 year old male is scheduled for reconstructive surgery on his knee in 6 weeks. He is begun on a naloxone patch, 200 mg, which is formulated to be rapidly absorbed over 6-8 hours as described above, on a nightly basis. To reduce the anxiety that this induces he is given an anxiolytic agent, diazepam (1-5 mg) at night along with the naloxone patch. After 2 weeks of this dose, the naloxone is increased to 400 mg on a nightly basis. The anxiolytic agent is used if needed. After yet an additional 2 weeks the naloxone is increased to 600-800 mg on a nightly basis. On the night of surgery and for several nights in the peri-operative period no naloxone is given. The patient is given only standard post-operative pain medications such as morphine and codeine. The doses of these substances are significantly reduced compared to the average individual undergoing this type of surgery, due to the up regulation of this patient's endorphin system. In an alternative method, after the first 2 weeks of naloxone treatment, the same patient is given a specific mu receptor antagonist along with the increasing dose of naloxone, in order to enhance the up regulation of pain-modulating mu receptors.

The methods of the invention may be used with mu and/or delta antagonists to elevate a patient's mood in the treatment of depression and related conditions. At first, non-specific opiate receptor antagonists (e.g., naloxone) may be administered to induce a counteradaptive response. Later in the treatement, it may be desirable to administer a specific delta opiate receptor antagonist because delta opiate receptors are strongly linked to mood. Of course, mu opiate receptor antagonists could be used, especially when chronic pain is associated with the depressed mood. When treating an already-depressed patient, the skilled artisan will closely monitor the patient for ill effects due to any acute worsening of mood due to antagonist-receptor binding.

In an example of a method of treating a depressed patient using the methods of the present invention, a 35 year old male with a diagnosis of clinical depression has had poor response and side effects with conventional antidepressant agents. He is especially consulted on the potential for temporary worsening of the depressed state, including suicidal tendencies. In-patient treatment in a hospital or appropriate mental institution is considered at the onset of therapy for higher risk potentially suicidal patients. After this is worked out, he is started on counteradaptation therapy protocol with the non-specific opiate antagonist naloxone. A transmucosal naloxone formulation is started prior to going to sleep, using a loading dose of 20 mg. A 30 mg transdermal dose, formulated to be absorbed over 6 hours, is applied at the same time. This 50 mg per 8 hour dose is given for two weeks. At two weeks the transmucosal dose is increased to 50 mg. The 6 hour transdermal dose is 50 mg, for a total of 100 mg. This dose is given for one month. Now, at 6 weeks after treatment had begun, the loading dose is 100 mg transmucosal and 100 mg transdermal over 6 hours. After another 4-6 weeks this is increased to 250 mg loading dose and 250 mg over 6 hours for a total 500 mg. After another on to two months this is increased to 500 mg loading dose and 500 mg over the next 6 hours. After another one or two or three months this is increased to a 1000 mg loading dose and a 1000 mg 6 hour transdermal dose. The maximal can stay for a long period of time at this 2000 mg total dose. Or it can continue to increase to 2,500, or 3,000 or 4,000 mg over the ensuing year or more. The maximal dose comes to a plateau once there is a good clinical response or once the side effects become too great or if there is an elevation of liver function enzymes on a blood test. The maximum tolerable dose is then given for an extended period of time for maintenance therapy. If and when therapy is stopped the patient is carefully monitored for any signs of recurrence of the mood disorder.

An option for the above-described patient is to add a delta opiate receptor antagonist, along with the naloxone after the first 6 weeks to 3 months of treatment. The naloxone dose may continue to be increased or it may level off earlier when combined with the delta antagonist. A non-peptide delta opiate receptor antagonist, such as naltrindole, natriben, or one of the agents discussed above, could be used. A peptide delta antagonist, such as ICI-154,129 or ICI-174,864 peptide, could also be used. The starting dose for naltrindole is larger than that for naloxone. It may be as high as 10 mg/kg/administration. Naltrindole may be given as a transdermal compound or using any other effective formulation.

The main consideration is the dosing for people with significant depression who may be at risk for suicide if the initial doses are too large. In a desirable embodiment of the invention, people with clinical depression, because they are suicidal risks, should either not be treated or treated at an in-patient hospital or appropriate institution in order to better monitor the patient. These patients are dosed at relatively lower doses at the beginning of treatment and that the increase in dose is done at a slower rate. Thus, for the depressed patients treatment may need to be started with a loading of only 10 mg of naloxone, with 10 or 20 mg to be absorbed over the ensuing 6 hours, for a total starting dose of 30 mg. Similarly, the increase in dose after 2 weeks is more gradual than for the example above. At 2 weeks one would give 20 mg as a loading dose and 20-40 mg over the ensuing 6 hours. This gradual increase is continued for as many months as is needed to obtain a maximal clinical response.

### The Dynorphin System

According to another embodiment of the invention, the neurotransmitter system is the dynorphin system, which includes dynorphins as neurotransmitters. Dynorphins are a class of endorphin compounds that bind preferentially to kappa receptors. Dynorphins generally have the opposite effect from the endorphins; their binding to kappa receptors is generally associated with a worsening of mood.

When the neurotransmitter system is the dynorphin system, the type of receptor is kappa receptors, which are generally positively linked to undesirable mental, neurological and physiological conditions. Kappa receptors are associated primarily with dysphoria when stimulated. The ligand is a kappa receptor agonist, and the counteradaptation causes a down-regulation of the dynorphin system. The counteradaptation may be, for example, a decrease in the biosynthesis or release of dynorphins at receptor terminals and/or by the pituitary gland; a decrease in the number of the receptors and/or dynorphin binding sites on the receptors; a decrease in the sensitivity of the receptors to binding by mu and/or delta receptor agonists and/or dynorphins; or any combination thereof. The counteradaptation may also up-regulate D2 (dopamine) receptors, which are negatively linked to depression.

A variety of kappa receptor agonists may be used in the present invention. For example, the kappa receptor agonist may be a peptide-based agonist, such as dynorphin [Dynorphin [A1-17], H-TYR-GLY-GLY-PHE-LEU-ARG-ARG-ILE8-ARG-PRO-LYS-LEU-LYS-TRP-ASP-ASN-GLN-OH] and all active peptide fragments and analogues thereof or a pharmaceutically acceptable salt, or carrier thereof. For example, the kappa receptor agonist may be the active C-terminal fragment of dynorphin A(1-8), or a pharmaceutically accepted salt or carrier thereof.

The kappa receptor agonist may also be non-peptidic. For example, the kappa receptor agonist may be a nonbenzomorphan; enadoline; PD117302; CAM569; PD123497; GR 89,696; U69,593; TRK-820; trans-3,4-dichloro-N-methyl-N-[1-(1-pyrrolidinyl)cyclohexyl]benzene-acetamide; asimadoline (EMD-61753); benzeneacetamide; thiomorpholine; piperidine; benzo[b]thiophene-4-acetamide; trans-(+/−)-(PD-117302); 4-benzofuranacetamide (PD-129190); 2,6-methano-3-benzazocin-8-ol (MR-1268); morphinan-3-ol (KT-90); GR-45809; 1-piperazinecarboxylic acid (GR-89696); GR-103545; piperzaine; GR-94839; xorphanl; benzeneacetamide (RU-49679); fedotozine; benzeneacetamide (DuP-747); HN-11608; apadoline (RP-60180); spiradoline mesylate; benzeneacetamide trans-U-50488 methane sulfate; 3FLB; FE200665; FE200666; an analogue of MPCB-GRRI or MPCB-RRI; benzomorphan kappa opioids, such as bremazocine and ethylketocyclazocine; or a pharmaceutically-accepted salt or carrier thereof.

The kappa receptor agonist may be U50,488 (trans-3,4-dichloro-N-[2-(1-pyrrolidinyl)cyclohexyl]benzeacetamide); spiradoline (U62,066E); Enadoline [(5R)-5α,7α,8β)-N-methyl-N-[7-(1-pyrrolidinyl)-1-oxzspiro[4,5]dec-8-yl]-4-benzofuranacetamide monohydrochloride] or PD117302 [(±)-trans-N-methyl-N-[2-(1-pyrrolidinyl)-cyclohexyl]benzo[b]thiophene-4-acetamide monohydrochloride] and their respective (+)-isomers (CAM569 and PD123497) (Parke-Davis Research Unit, Cambridge, UK), each of which are highly selective arylacetamide kappa opioids; GR89,696 (4-[(3,4-dichlorophenyl)acetyl]-3-(1-pyrrolidinylmethyl)-1-piperazinecarboxylic acid methyl ester fumarate), a prototypical arylacetamide developed from the structure of U50,48811 having high efficacy as a K₁ agonist; U69,593 [(5α,7α,8β)-(+)-N-methyl-N-(7-(1-pyrrolidinyl)-1-oxaspiro[4,5]dec-8-yl)benzeneacetamide], a kappa agonist with K₁ selectivity; TRK-820 ((−)-17-cyclopropylmethyl-3,14b-dihydroxy-4,5a-epoxy-6b-[N-methyl-trans-3-(3-furyl)acrylamide]morphinan hydrochloride) (Toray Industries, Inc. Japan), a potent kappa agonist with pharmacological properties different from those produced by K₁ receptor agonists; tifluadom, a benzodiazepine kappa agonist (Sandoz, Inc., Princeton, N.J.); or trans-3,4-dichloro-N-methyl-N-[1-(1-pyrrolidinyl)cyclohexyl]benzeneacetamide, a kappa agonist described in U.S. Pat. No. 4,758,562.

Kappa receptor agonists are also described in U.S. Pat. Nos. 5,051,428; 5,965,701; 6,146,835; 6,191,126; 6,624,313; 6,174,891; 6,316,461; 6,440,987; 4,758,562; 6,583,151, each of which is incorporated herein by reference in its entirety.

The initial dosage of the kappa receptor agonist is desirably high enough to induce a counteradaptive effect, but not so high as to cause the patient intolerable direct effects. For example, the initial dosage of the kappa receptor agonist may be equivalent to between 0.0005 and 0.05 mg/kg/administration of dynorphin; between 5 and 700 mg/administration of enadoline; between 1 and 500 μg/administration of FE 20665; between 0.5 and 100 μg/administration; between 0.01 and 1 mg/kg/administration of U69,593; between 0.05 and 5 mg/kg/administration of TRK 820; between 0.01 and 1 mg/kg/administration U 50 488 or between 0.01 and 1 mg/kg/administration of PD 117302. Desirably, the initial dosage of the kappa receptor agonist is equivalent to between 0.005 and 0.02 mg/kg/administration of dynorphin; between 100 and 500 mg/administration of enadoline; between 3 and 100 μg/administration of FE 20665; between 1 and 80 μg/administration of FE 20666; between 0.1 and 0.7 mg/kg/administration of U69,593; between 0.5 and 3 mg/kg/administration of TRK 820; between 0.5 and 7 mg/kg/administration U 50 488 or between 0.1 and 0.7 mg/kg/administration of PD 117302.

In another embodiment of the invention, the kappa receptor agonist is Salvinorin A. Salvinorin A is a neoclerodane diterpene compound, which is a very powerful hallucinogen that has recently been found to have kappa agonist activity. It represents the only known non-nitrogenous kappa agonist compound. It is the main active ingredient of the plant S. divinorum (Diviner's sage), a rare member of the mint family. It has been used for many centuries by the Mazatec people of Oaxaca, Mexico in traditional spiritual practices. The initial dose of Salvinorin A is desirably between 5 and 50 μg/administration, and the maximum dose is desirably 5000 μg/administration. The Salvornin A may be administered transmucosally, or as a slow-release formulation, desirably over a period between two and six hours in duration.

In certain embodiments of the invention, it may be desirable to administer both a peptidic kappa receptor agonist and a non-peptidic kappa receptor agonist. The two types of agonist may be administered substantially simultaneously, or sequentially.

Peptidic kappa receptor agonists may be administered, for example, intravenously, transdermally, or transmucosally, as described above with respect to other peptidic ligands. As described above with respect to naloxone, it may be desirable to use transmucosal administration (to achieve a high level of ligand-receptor binding quickly) along with transdermal administration (to provide extended ligand-receptor binding).

Because the body develops a tolerance to anti-opiates about eight days after first administration, it may be desirable to increase the dosage of the kappa receptor agonist with time. For example, it may be desirable to increase the dosage with a period of between a week and two weeks.

In an example of a method of the present invention using Salvinorin A, the initial dose of Salvinorin A is low in order to decrease potential side effects. A dose between 5 μg-50 μg is the starting dose. After 2-4 weeks this is increased by a certain percent. The increase could be as small as 5-10% or 50-100% or more. Generally, a doubling of the initial dose is recommended. Thus, after 2-4 weeks the individual is given 20-100 μg of Salvinorin A. This increase in dose is continued every two, four, six or eight weeks. It may also continue to increase on a quarterly, semiannual or annual basis. Doses of 200 μg may produce increasing dysphoric effects. This occurs with acute administration. With chronic gradual increase in dose the side effects would be gradually muted. With chronic gradual increase in dosing the maximum dose of Salvinorin A is 1000 μg to 5000 μg or more.

In an example of the method of the present invention using a dynorphin analogue, a rectal suppository (transmucosal) formulation is used. The initial dose is high enough in order to induce a counteradaptive response, but low enough to minimize dysphoric effects of agonist-receptor binding. There is a two-part construct of the suppository. The outer covering is rapidly dissolved and allows for an initial rapid absorption of the kappa receptor agonist compound. The second layer is gradually broken down in order to slowly release additional kappa receptor agonist, which is gradually absorbed. This results in a continuous, slow-release absorption of the peptide kappa receptor agonist compound. It is designed to last for 6-8 hours of gradual absorption such that there is 6-8 hours of kappa receptor binding, at which time the counteradaptive response is induced. This rectal suppository is given on a daily (nightly) basis. After 2-4 weeks the dose is doubled. This dose is then given for an additional 2-4-6-8 weeks. The dose is intermittently increased until the development of side effects prevents a further increase. As the dose is increased the time interval for increasing the dose is lengthened, such that several months may pass before increasing the dose. In addition, once higher doses are used the increase is less dramatic, such that only 5-10% increases are given, rather than the initial doubling of the dose.

Enadoline is a non-peptidic kappa receptor agonist. It has pharmaceutical activity when taken as an oral dose at 1-10 mg/kg. In an example of a method of the present invention using enadoline, an initial dose of 100-200 mg is administered daily just prior to the patient's going to bed. After 2-4 weeks the dose is increase to 200-500 mg. After another 2-4 weeks the dose is increased to 500-1000 mg. After another two, four, eight weeks or more, it is increased to 1500-2000 mg. The dose is increased as long as side effects do not become uncontrollable.

Desirably, a kappa receptor antagonist is not administered during the first time period associated with each administration. In certain embodiments of the invention, however, a kappa receptor antagonist is administered during one or more of the second time periods. Representative kappa receptor antagonists include the compounds described in U.S. Pat. Nos. 5,025,018; 5,922,887; and 6,284,769. For the compounds described in 5,025,018, a suitable dosage includes 0.1 to 10 mg/administration per day; for U.S. Pat. No. 6,284,769, suitable dosages include 0.1 to 500 mg/administration.

The dynorphin neurotransmitter system and its kappa receptors are positively linked to a wide variety of undesirable mental and neurological conditions. Examples of such conditions include pain, mood disorders, eating disorders, anxiety disorders, motivational problem, substance abuse disorders, insufficient motivation or performance, pain that is expected to occur in the future (e.g., due to a future operation or future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, generalized anxiety state, panic disorders, Tourette's Syndrome, hysteria sleep disorders, breathing-related sleep disorders, lack of motivation due to learning or memory problems, abuse of substances such as narcotics, alcohol, nicotine, stimulants, anxiolytics, CNS depressants, hallucinogens and marijuana, and insufficient motivation or preparation for a desired mental or physical activity such as physical training, athletics, studying or testing. The down-regulation of the dynorphin system desirably causes a therapeutic benefit with respect to the undesirable mental, neurological or physiological condition.

### The Serotonin System

According to another embodiment of the invention, the neurotransmitter system is the serotonin system which includes serotonin as a neurotransmitter. Serotonin is a monoamine neurotransmitter. Low serotonin levels are associated with depression. The counteradaptation causes an up-regulation of the serotonin system.

Numerous serotonin receptors (at least 14) have been identified. The greatest concentration of serotonin (90%) are located in the gastrointestinal tract. Most of the remainder of the body's serotonin is found in platelets and the central nervous system (CNS). The effects of serotonin are noted in the cardiovascular system, the respiratory system and the intestines. Vasoconstriction is a typical response to serotonin.

The function of serotonin is exerted upon its interaction with specific receptors. Several serotonin receptors have been cloned and are identified as 5HT₁, 5HT₂, 5HT₃, 5HT₄, 5HT₅, 5HT₆, and 5HT₇. Within the 5HT₁ group there are subtypes 5HT_{1A}, 5HT_{1B}, 5HT_{1D}, 5HT_{1E}, and 5HT_{1F}. There are three 5HT₂ subtypes, 5HT_{2A}, 2HT_{2B}, and 5HT_{2C} as well as two 5HT₅ subtypes, 5HT₅ₐ and 5HT_{5B}. Most of these receptors are coupled to G-proteins that affect the activities of either adenylate cyclase or phospholipase Cg. The 5HT₃ class of receptors are ion channels

Some serotonin receptors are presynaptic and others postsynaptic. The 5HT_{2A} receptors mediate platelet aggregation and smooth muscle contraction. The SHT_{2c} receptors are suspected in control of food intake as mice lacking this gene become obese from increased food intake and are also subject to fatal seizures. The 5HT₃ receptors are present in the gastrointestinal tract and are related to vomiting. Also present in the gastrointestinal tract are 5HT₄ receptors where they function in secretion and peristalsis. The 5HT₆ and SHT₇ receptors are distributed throughout the limbic system of the brain and the 5HT₆ receptors have high affinity for antidepressant drugs.

The most common serotonin receptors that are associated with mood and depression are the 1^{st} and 2^{nd} ones, most especially the 5HT_{1A} receptors.

When a serotonin neuron is stimulated to fire, serotonin is released into the synapse. Some serotonin molecules cross the synapse and bind to the post-synaptic receptor, which then causes firing of the post-synaptic serotonin neuron. Binding of serotonin to the post-synaptic serotonin neuron causes its activation, which leads to a series of neural events that is associated with a generally good mood.

When serotonin is released into the synaptic cleft only a portion of the serotonin actually binds to post-synaptic receptors. The majority of serotonin molecules are removed from the synapse by a reuptake mechanism. Some of this serotonin is degraded by monoamine oxidases, enzymes that degrade both serotonin and norepinephrine.

The third target of serotonin molecules are the pre-synaptic auto-receptors. The pre-synaptic autoreceptors are inhibitory receptors. The pre-synaptic autoreceptors act in a feedback inhibition loop that functions as a control mechanism for neurotransmitter release. A feedback inhibition loop is a common manner by which the body controls the activation of neurons. When they are bound by sertonin, or an agonist, they inhibit the further release of sertonin into the synapse. Pre-synaptic autoreceptors are termed 5HT_{1A} and 5HT_{1B} pre-synaptic autoreceptors. 5HT_{1A} autoreceptors inhibit the tonic release of serotonin. 5HT_{1B} autoreceptors are thought to inhibit the evoked release and synthesis of serotonin.

When the neurotransmitter system is the serotonin system, the type of receptor may be, for example, serotonin pre-synaptic autoreceptors such as 5HT_{1A} autoreceptors or 5HT_{1B} autoreceptors. In such cases, the ligand is a serotonin pre-synaptic autoreceptor agonist, and the undesirable mental, neurological or physiological condition is positively linked to the receptors. The counteradaptation may be, for example, an increase in the biosynthesis and/or release of serotonin at the synaptic cleft; a decrease in the reuptake of serotonin; a decrease in the number of serotonin pre-synaptic autoreceptors; a decrease in the sensitivity of the serotonin pre-synaptic autoreceptors to serotonin and/or serotonin pre-synaptic autoreceptor agonists; an increase in the number of serotonin post-synaptic receptors; an increase in the sensitivity of the serotonin post-synaptic receptors to serotonin or serotonin post-synaptic receptor agonists; or any combination thereof.

A variety of serotonin pre-synanptic autoreceptor agonists may be used in the methods of the present invention. For example, the serotonin pre-synaptic autoreceptor agonist may be EMD-68843, buspirone, gepirone, ipsapirone, tandospirone, Lesopitron, zalospirone, MDL-73005EF, or BP-554.

The initial dosage of the serotonin pre-synaptic autoreceptor agonist is desirably high enough to induce a counteradaptive effect, but not so high as to cause the patient intolerable direct effects. For example, the initial dosage of the serotonin pre-synaptic autoreceptor agonist may be equivalent to between 1 and 400 mg/administration of EMD-68843, between 1 and 500 mg/administration buspirone, between 1 and 500 mg/administration lesopitron, between 1 and 500 mg/administration gepirone, between 5 and 500 mg tandospirone, or between 1 and 200 mg zalospirone. Desirably, the initial dosage of the serotonin pre-synaptic autoreceptor agonist is equivalent to between 10 and 100 mg/administration of EMD-68843, between 10 and 100 mg/administration buspirone, between 10 and 200 mg/administration lesopitron, between 10 and 100 mg/administration gepirone, between 20 and 200 mg tandospirone, or between 10 and 100 mg zalospirone.

Desirably, a serotonin pre-synaptic autoreceptor antagonist is not administered during the first time period associated with each administration. In certain embodiments of the invention, however, a serotonin pre-synaptic autoreceptor antagonist is administered during one or more of the second time periods. Representative serotonin pre-synaptic autoreceptor 5HT1A agonists and antagonists include Elazonan, AR-A2 (AstraZeneca, London, UK); AZD-1134 [AstraZeneca, London, UK); Pindolol, as well as compounds described in U.S. Pat. No. 6,462,048; 6,451,803; 6,627,627; 6,602,874; 6,277,852; and 6,166,020, incorporated by reference in their entirety.

In another embodiment of the invention, the type of receptor is serotonin post-synaptic receptors, such as 5HT₁ receptors; 5HT₂ receptors; 5HT₃ receptors; 5HT₄ receptors; 5HT₅ receptors; 5HT₆ receptors; 5HT₇ receptors; or receptors of a subtype thereof. The ligand is a serotonin post-synaptic receptor antagonist. Undesirable mental, neurological or physiological conditions are generally negatively linked with these receptors. The counteradaptation may be an increase in the biosynthesis and/or release of serotonin at thesynaptic cleft; a decrease in the reuptake of serotonin; an increase in the number of serotonin post-synaptic receptors; an increase in the sensitivity of the serotonin post-synaptic receptors to serotonin and/or serotonin post-synaptic receptor agonists; a decrease in the number of serotonin pre-synaptic autoreceptors; a decrease in the sensitivity of the serotonin pre-synaptic autoreceptors to serotonin and/or serotonin pre-synaptic autoreceptor agonists; or any combination thereof.

A variety of compounds may be used as the serotonin post-synaptic receptor antagonists in the methods of the present invention. For example, the serotonin post-synaptic receptor antagonists may be (S)-WAY-100135, WAY-100635, buspirone, gepirone, ipsapirone, tandospirone, Lesopitron, zalospirone, MDL-73005EF, or BP-554. If desired, an SSRI maybe administered either simultaneously or sequentially with the aforementioned serotonin modulating agents. This is advantageous as both SSRI and agonist pre-synaptic counteradaptive therapy result in a down regulation of the pre-synaptic receptors. The SSRI effect is thus magnified by such a counteradaptive effect. Second, any down regulation of the post synaptic serotonin receptors that may occur with SSRI therapy is counterbalanced by post synaptic antagonist counteradaptive therapy.

The initial dosage of the serotonin post-synaptic antagonist is desirably high enough to induce a counteradaptive effect, but not so high as to cause the patient intolerable direct effects. For example, the initial dosage of the serotonin post-synaptic receptor antagonist is equivalent to between about 0.01 and 5 mg/kg/administration of WAY-100635. Desirably, the initial dosage of the serotonin post-synaptic receptor antagonist is equivalent to between about 0.025 and 1 mg/kg/administration of WAY-100635.

The serotonin post-synaptic receptor antagonist may be administered in combination with a serotonin pre-synaptic autoreceptor agonist, such as those described above. Further, when conventional anti-depressant agents that bind at the serotonin post-synaptic receptors are given in combination with a serotonin pre-synaptic autoreceptor agonist, its efficacy can be greatly increased because the serotonin post-synaptic receptors have increased in number and/sensitivity through the counteradaptation.

In certain desirable embodiments of the invention, the serotonin post-synaptic antagonist itself is also a serotonin pre-synaptic autoreceptor agonist. It may also be desirable to administer a norepinephrine pre-synaptic alpha-2 adrenergic receptor agonist and/or a norepinephrine post-synaptic adrenergic receptor antagonist (as described below) in combination with the serotonin post-synaptic antagonist or serotonin pre-synaptic autoreceptor agonist.

Desirably, a serotonin post-synaptic receptor agonist is not administered during the first time period associated with each administration. In certain embodiments of the invention, however, a serotonin post-synaptic receptor agonist is administered during one or more of the second time periods. Representative serotonin post-synaptic receptor agonists include BIMT 17 (1-[2-[4-(3-trifluoromethyl phenyl)piperazin-1-yl]ethyl]benzimidazol-[1H]-2-one), dose: 1-10 mg/kg (i.v. or transdermal, SQ, etc.). See Borsini, F, et al., Archives of Pharmacology, 352(3); September, 1995:283-290.] A suitable dosage range includes 1 to 10 mg/kg/administration of BIMT 17 (via iv, transdermal, or SQ).

Serotonin post-synaptic receptors are generally negatively linked, and serotonin pre-synaptic autoreceptors are generally positively linked to a wide variety of undesirable mental and neurological conditions. Examples of such conditions include pain, mood disorders, eating disorders, anxiety disorders, obsessive-compulsive disorders, motivational problem, substance abuse disorders, insufficient motivation or performance, pain that is expected to occur in the future (e.g., due to a future operation or future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, generalized anxiety state, panic disorders, Tourette's Syndrome, hysteria sleep disorders, breathing-related sleep disorders, lack of motivation due to learning or memory problems, abuse of substances such as narcotics, alcohol, nicotine, stimulants, anxiolytics, CNS depressants, hallucinogens and marijuana, and insufficient motivation or preparation for a desired mental or physical activity such as physical training, athletics, studying or testing. The up-regulation of the serotonin system desirably causes a therapeutic benefit with respect to the undesirable mental, neurological or physiological condition.

### The Norepinephrine System

In another embodiment of the invention, the neurotransmitter system is the norepinephrine system which includes norepinephrine as a neurotransmitter, and the counteradaptation causes an up-regulation of the norepinephine system.

Norepinephrine is a catecholamine that, along with epinephrine, acts as a neurotransmitter in the central nervous system. There are two types of adrenoreceptors, alpha and beta. There are in addition, at least ten different subtypes of adrenoreceptors. Norepinephrine generally is more potent at sites where sympathetic neurotransmission is excitatory and is mediated through alpha receptors. Alpha receptors have two main subclasses, alpha1 and alpha2.

Norepinephrine acts a neuromodulator in the central nervous system. The central nervous system actions of NE are most notable when it modulates excitatory or inhibitory inputs, rather than its effects on the activity of post-synaptic targets, in the absence of other inputs. Norepinephrine transmission and control is similar to that for serotonin. A reuptake mechanism is present that removes the majority of norepinephrine after its release into the noradrenergic synapse. There are pre-synaptic inhibitory autoreceptors known as alpha-2 adrenergic receptors.

When the neurotransmitter system is the norepinephrine system, the type of receptor may be, for example, norepinephrine pre-synaptic alpha-2 adrenergic receptors. In such cases, the ligand is a norepinephrine pre-synaptic alpha-2 adrenergic receptor agonist. Undesirable mental, neurological or physiological conditions are generally positively linked to the receptors. The counteradaptation may be an increase in the biosynthesis and/or release of norepinephrine at the synaptic cleft; a decrease in reuptake of norepinephrine; a decrease in the number of norepinephrine pre-synaptic alpha-2 adrenergic receptors; a decrease in the sensitivity of the norepinephrine pre-synaptic alpha-2 adrenergic receptors to norepinephrine and/or norepinephrine pre-synaptic alpha-2 adrenergic receptor agonists; an increase in the number of norepinephrine post-synaptic adrenergic receptors; an increase in the sensitivity of the norepinephrine post-synaptic adrenergic receptors to norepinephrine and/or norepinephrine post-synaptic adrenergic receptor agonists; or any combination thereof.

A variety of compounds may be used as the norepinephrine pre-synaptic alpha-2 adrenergic receptor agonists in the methods of the present invention. For example, the norepinephrine pre-synaptic alpha-2 adrenergic receptor agonist may be clonidine, guanfacine, lofexidine, detomidine, dexmedetomidine, mivazerol, or alpha-methylnoradreniline.

The initial dosage of the norepinephrine pre-synaptic alpha-2 adrenergic receptor agonist is desirably high enough to induce a counteradaptive effect, but not so high as to cause the patient intolerable direct effects. For example, the initial dosage may be equivalent to between 0.1 and 10 μg/kg/administration of clonidine, between 0.01 and 10 mg/administration guanfacine, between 0.01 and 1 mg/administration lofexidine, between 1 and 100 μg/kg/administration detomidine, between 0.05 and 5 μg/kg/administration dexmedetomidine, between 0.05 and 10 μg/kg/administration mivazerol, or between 5 and 500 ng/kg/administration of alpha-methylnoradreniline. Desirably, the initial dosage is equivalent to between 0.1 and 0.5 mg/administration of clonidine, between 0.1 and 5 mg/administration guanfacine, between 0.05 and 0.5 mg/administration lofexidine, between 10 and 80 μg/kg/administration detomidine, between 0.1 and 3 μg/kg/administration dexmedetomidine, between 0.5 and 5 μg/kg/administration of mivazerol, or between 10 and 100 ng/kg/administration of alpha-methylnoradreniline.

Desirably, a norepinephrine pre-synaptic alpha-2 adrenergic receptor antagonist is not administered during the first time period associated with each administration. In certain embodiments of the invention, however, an norepinephrine pre-synaptic alpha-2 adrenergic receptor antagonist is administered during one or more of the second time periods. A suitable non-limiting example of a pre and postsynaptic A2AR antagonist includes mirtazapine.

According to another embodiment of the invention, the type of receptor is norepinephrine post-synaptic adrenergic receptors, such as alpha receptors, beta receptors, or receptors of a subtype thereof. In such cases, the ligand is a norepinephrine post-synaptic adrenergic receptor antagonist. Undesirable mental, neurological or physiological conditions are generally negatively linked to the norepinephrine post-synaptic adrenergic receptors. The counteradaptation may be an increase in the biosynthesis or release of norepinephrine at the synaptic cleft; a decrease in the reuptake of norepinephrine; an increase in the number of norepinephrine post-synaptic adrenergic receptors; an increase in the sensitivity of the norepinephrine post-synaptic adrenergic receptors to norepinephrine and/or norepinephrine post-synaptic adrenergic receptor agonists; a decrease in the number of norepinephrine pre-synaptic alpha-2 adrenergic receptors; a decrease in the sensitivity of the norepinephrine pre-synaptic alpha-2 adrenergic receptors to norepinephrine and/or norepinephrine pre-synaptic alpha-2 adrenergic receptor agonists; or any combination thereof.

A variety of compounds may be used as the norepinephrine post-synaptic adrenergic receptor antagonists in the methods of the present invention. For example, the norepinephrine post-synaptic adrenergic receptor antagonist may be idazoxan, SKF 104078, or SKF 104856. The initial dosage of the norepinephrine post-synaptic adrenergic receptor antagonist is desirably high enough to induce a counteradaptive effect, but not so high as to cause the patient intolerable direct effects. For example, the initial dosage may be equivalent to between 0.5 and 100 mg/administration of idazoxan. Desirably, the initial dosage is equivalent to between 5 and 50 mg/administration of idazoxan.

Desirably, a norepinephrine post-synaptic adrenergic receptor agonist is not administered during the first time period associated with each administration. In certain embodiments of the invention, however, a norepinephrine post-synaptic adrenergic receptor agonist is administered during one or more of the second time periods.

The norepinephrine post-synaptic adrenergic receptor antagonist may be administered in combination with a norepinephrine pre-synaptic alpha-2 adrenergic receptor agonist, such as those described above. Further, when conventional anti-depressant agents that bind at the norepinephrine post-synaptic adrenergic receptors are given in combination with a norepinephrine pre-synaptic alpha-2 adrenergic receptor agonist, its efficacy can be greatly increased because the norepinephrine post-synaptic adrenergic receptors have increased in number and/sensitivity through the counteradaptation.

In certain desirable embodiments of the invention, the norepinephrine post-synaptic adrenergic receptor antagonist itself is also an norepinephrine pre-synaptic alpha-2 adrenergic receptor agonist. It may also be desirable to administer a serotonin post-synaptic antagonist and/or a serotonin pre-synaptic autoreceptor agonist (as described above) in combination with the norepinephrine pre-synaptic alpha-2 adrenergic receptor agonist or norepinephrine post-synaptic adrenergic receptor antagonist.

Norepinephrine post-synaptic adrenergic receptors are generally negatively linked, and norepinephrine pre-synaptic alpha-2 adrenergic receptors are generally positively linked to a wide variety of undesirable mental and neurological conditions. Examples of such conditions include pain, mood disorders, eating disorders, anxiety disorders, obsessive-compulsive disorders, motivational problem, substance abuse disorders, insufficient motivation or performance, pain that is expected to occur in the future (e.g., due to a future operation or future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, generalized anxiety state, panic disorders, Tourette's Syndrome, hysteria sleep disorders, breathing-related sleep disorders, lack of motivation due to learning or memory problems, abuse of substances such as narcotics, alcohol, nicotine, stimulants, anxiolytics, CNS depressants, hallucinogens and marijuana, and insufficient motivation or preparation for a desired mental or physical activity such as physical training, athletics, studying or testing. The up-regulation of the norepinephrine system desirably causes a therapeutic benefit with respect to the undesirable mental, neurological or physiological condition.

### The CRF System

The hypothalamic-pituitary-adrenal (HPA) axis is the regulatory mechanism by which the body responds to stress. The hypothalamus is a general control center for many of the body's hormones. In response to stress the hypothalamus releases corticotropin releasing factor (CRF, also known as corticotropin releasing hormone), which reaches the anterior pituitary gland and induces changes that release the hormone ACTH (adrenocorticotropic hormone) and beta-endorphin into the circulation. ACTH reaches the adrenal glands, which are located adjacent to the kidneys, and simulates the release of cortisol. Cortisol release into the circulation initiates a series of metabolic effects that alleviate the harmful effects of stress. There is also a negative feedback to both the hypothalamus and the anterior pituitary which shuts off further cortisol release.

Besides CRF release from the hypothalamus, CRF is present in many other areas of the cortex. When it is released from the hypothalamus it acts as a hormone. In the cortex the CRF molecule acts as a neurotransmitter. Neurotransmitter effects of the CRF results in some of the behavioral effects that are common in depression. Some of these effects are due to the CRF effect on other neurotransmitter systems, such as the serotonin and norepinephrine (NE) systems. The CRF relation to depression is thus quite complex and relates to its effects on the HPA axis as well as direct effects on the brain and on other neurotransmitter systems.

CRF is a 41 amino acid peptide. It was first isolated and sequenced by Vale in 1981 (Vale W, Spiess J, Rivier C, Rivier J (1981): Characterization of a 41-residue ovine hypothalamic peptide that stimulates secretion of corticotropin and β-endorphin. Science 213:1394-1397). The sequence of CRF has been determined in a variety of species, including sheep, man, rats, pigs, goats and cows. In all species, CRF is a 41-amino acid residue single chain polypeptide. Rat and human CRF are identical to one another and differ from ovine CRF by seven amino acid residues. All three CRFs have close amino acid homology and share some biologic properties with sauvagine, a 40-amino acid peptide that exists in frog skin, and urotensin I, a 41-amino acid peptide derived from fish urophysis. Caprine and ovine CRF are identical and differ from bovine CRF by one amino acid. Porcine CRF more closely resembles rat/human CRF. CRF, and related peptides, are amidated at their carboxy terminal; CRF COOH-terminal-free acid has less than 0.1% of the potency of native CRF, suggesting the importance of amidation to the biological activity of the peptide. Studies to determine the solution structure of CRF using proton nuclear magnetic resonance spectroscopy suggest that human CRF comprises an extended N-terminal tetrapeptide connected to a well-defined α-helix between residues 6 to 36. An α-helical oCRF(9-41) is an antagonist of CRF, which underscores the necessity of the α-helical conformation for receptor binding and biological activity. (Errol B. De Souza and Dimitri E. Grigoriadis, Corticotropin-Releasing Factor: Physiology, Pharmacology and Role in Central Nervous System and Immune Disorders, Psychopharmacology—Fourth Generation of Progress, 2000, http://www.acnp.org/g4/GN401000049/CH049.html)

Mood, mood disorders and related conditions are a result of a complex web of central nervous system events that interrelate many neurotransmitter systems. A most common mood disorder is depression. Depression is a clinical diagnosis with numerous somatic and mental symptoms, which is due to an alteration of numerous neurotransmitter systems. Beisdes the CRF system other systems associated with depression are the norepinephrine, serotonin, substance P, dynorphin (kappa receptors), and the endogenous endorphin (mu and delta opiate receptors) systems. Further, these neurotransmitter systems are also related to a whole host of other undesirable mental and neurological conditions, including bipolar disorders, obsessive-compulsive disorders, anxiety, phobias, stress disorders, substance abuse, sexual disorders, eating disorders, motivational disorders and pain disorders.

Stress is felt to be a major cause of depression and anxiety in the adult. Whether or not an individual actually becomes clinically depressed also depends on the genetic predisposition to depression and any major early life stressful situations (Lott, Psychiatric Times 1999 October; Vol. XVI, Issue 10.).

The HPA axis plays a major role in depression and anxiety disorders because of its role in stressful situations. It is well established that CRF levels are increased in depression (ie., melancholic depression, but not atypical depression—see below). Cortisol levels are also generally increased in depression. This is due to a hyper activity of the HPA axis, largely thought to be due to an impairment of the negative inhibition loop. In other words, the persistent release of CRF and its persistent elevation in the circulation, which may be a result of chronic stressful situations, is not inhibited by the elevated cortisol levels, as would occur in the normal, non-depressed individual.

Depression studies generally categorize all patients into one category. There are however different forms of depression, which tend to have different regulatory changes in the HPA axis. There are two notable subtypes of depression, melancholic and atypical. Melancholic depression is the most common form of depression. Melancholic patients are generally anxious, dread the future, lose responsiveness to the environment, have insomnia, lose their appetite (weight loss), and a diurnal variation with depression at its worst in the morning. Atypical patients are generally opposite in many of these areas. Atypical patients generally are lethargic, fatigued, hyperphagic (weight gain), hypersomnic, reactive to the environment, and show diurnal variation of depression that is at its best in the morning. (Gold, et al, “Organization of the stress system and its dysregulation in melancholic and atypical depression: high vs. low CRH/NE states.” Mol Psychiatry, 2002; 7(3):254-75.”)

Melancholic patients are characterized by a hyperactive central CRH system with over-activity of the HPA axis. On the other hand, atypical depression is characterized by a hypo-active central CRH system and an under-activity of the HPA axis. (Kasckow, J W, et al, “Corticotropin-releasing hormone in depression and post-traumatic stress disorder.” Peptides, 2001 May; 22(5):845-51.) Atypical depression is thought to be due to hyper-suppression of the IPA axis. It may be associated with an exaggerated negative feedback regulation of the HPA axis. (Levitan RD, “Low-dose dexamethasone challenge in women with atypical major depression: pilot study.” J Psych Neurosci, 2002 January; 27(1):47-51)

Post-traumatic stress disorder (PTSD) is a third category and it is also unique. PTSD is characterized by a hyperactive central CRH system as in melancholic depression, but there is under-activity of the EPA axis as in atypical depression. (Levitan, 2002)

Anxiety disorders have a different pattern of HPA axis changes than does melancholic depression. Depression is characterized by a hypercortisolemia, non-suppression after dexamethasone and a decrease in the number of glucocorticoid receptors. Anxiety is characterized by hypo-cortisolemia, super-suppression after dexamethasone and an increase in the number of glucocorticoid receptors. (Boyer, P, “Do anxiety and depression have a common pathophysiological mechanism?” Acta Psychiatr Scand Suppl 2000; (406):24-9)

Eating disorders are largely related to mood and/or stress. Dallman et al., (Chronic stress and obesity: a new view of “comfort food”. Proc Natl Acad Sci USA 2003 Sep. 30; 100(20):11696-701) demonstrate that, although rats generally decrease weight in response to chronic stress, chronic stress in humans induces either increased comfort food intake and body weight gain or decreased intake and body weight loss. As discussed above melancholic depression tends to be associated with poor appetite and weight loss and atypical depression tends to be associated with increased eating and weight gain.

Conventional strategies for treating neurotransmitter-linked conditions are centered on improving abnormally high or low levels of synaptic neurotransmitters. Conventional therapeutic agents work to directly regulate the functioning of the neurotransmitter systems. Such agents may be anxiolytic agents, hypnotic agents, or selective reuptake inhibitors, and include benzodiazepines (e.g., diazepam, lorazepam, alprazolam, temazepam, flurazepam, and chlodiazepoxide), TCAs, MAOIs, SSRIs (e.g., fluoxetine hydrochloride), NRIs, SNRIs, serotonin pre-synaptic autoreceptor antagonists, 5HT₁ agonist, GABA-A modulating agents, serotonin 5H_{2C} and/or 5H_{2B} modulating agents, beta-3 adrenoceptor agonists, NMDA antagonists, V1B antagonists, GPCR modulating agents, dynorphin antagonists, and substance P antagonists. CRF antagonists are thought to be the next class of antidepressants (Nielsen, Life Sci, 2006, Jan. 25; 78(9): 909-19.).

With respect to the CRF system, because of the different HPA axis changes with different types of depression, the response to pharmaceutical agents is expected to vary, depending on the type of depression or anxiety disorder that is being treated. Indeed, antidepressants that are effective in decreasing CRH production, ie. TCAs, have good efficacy for melancholic depression. These same agents are not particularly effective for the treatment of atypical depression, which is not associated with activation of the CRH-producing system, but rather to a decrease in CRH secretion. (Licinio, J, et al, “Role of corticotrophin releasing hormone 41 in depressive illness.” Baillieres Clin Endocrinol Metab, 1991 March; 5(1):51-8.)

Numerous studies have indicated the potential of CRF antagonists as antidepressants (O'brien, Hum Psychopharmacol, 2001, January; 16(1): 81-87 and Arborelius, et al, J Endocrinol 1999, 160: 1-12). Because they block the effects of a hyper-active CRF system, CRF antagonists are thought to be indicated for conditions that have a hyper-active CRF system, such as melancholic depression, rather than for atypical depression, which has a hypo-active CRF system.

Beta-endorphin is an endogenous opiate compound that is beneficial during a stress response. It reduces the sensation of pain. Endorphins are the result of an evolutionary mechanism which ensures that survival comes first, and recuperation comes later. Pain would ordinarily produce behaviors that would hurt the chances of survival. For instance, if an animal is attacked and stops to lick its wounds instead of fleeing away from its attacker, the animal's life is put in danger. But, fear triggers the release of endorphins which that inhibit the perception of pain. Furthermore, endorphins have an effect on the immune system that better helps ward off infection, which is advantageous, had the animal been injured.

Beta endorphin is closely associated with the HPA. In addition to its actions on areas in the cortex of the brain, such as improving pain response, beta endorphin also regulates the release of CRF through a negative inhibition at the hypothalamus. (FIG. X)

Beta endorphin and endogenous opioid peptides in general inhibit the HPA by decreasing the release of CRF (Burnett, J Affect Disord 1999 June; 53(3): 263-8.). This latter study demonstrates that there is decreased inhibitory opioid tone in depressed individuals. In other words there is a down regulation of the beta endorphin system in depressed individuals. This is corroborated by other studies which demonstrate that depression is associated with decreased circulating levels of beta endorphin (Cohen, A M J Psychiatry 1984; 141: 629-32., and Djurovic, Farmaco 1999 Mar. 31; 54(3): 130-3.).

Beta endorphins were also demonstrated to be increased in depressed individuals (Goodwin, J Affect Disord 1993 December; 29(4): 281-9). Although this finding seems to contradict the above studies, wehre endorphin levels are decreased in depression, this latter study is actually consistent with the above studies. This is explained as follows. In the latter study (Goodwin), beta endorphin was actually negatively correlated with the severity of depression symptoms, which is consistent with the fact that down regulation of beta endorphin is associated with depression, and the above studies (Cohen & Djurovic). The Goodwin study demonstrates that an acute psychosocial precipitant can increase beta endorphin levels acutely. This indicates that acute stressful events can cause such an increase in CRF that it can override the down regulated beta endorphin system. However, the Goodwin study also confirms the fact that there is a general down regulation of beta endorphin with depression, and that the severity of depression is inversely correlated with the levels of circulating beta endorphins, just as was demonstrated by the Cohen and Djurovic studies. In summary, although beta endorphins may be acutely elevated in depressed individuals who are subjected to a stressful precipitating event, on a daily steady state level depressed patients have lower than normal levels of circulating endorphins.

The Burnett (1999) study summarizes that some clinically depressed individuals may self-medicate with opiates. In those situations the opiates replace the ‘missing’ or down regulated endogenous endorphins. These self-administered opiates perform the same task as do endogenous endorphins, which are meant to inhibit the HPA, thereby decreasing CRF release.

Because hyperactivity of the HPA is associated with depression the use of CRF antagonists has recently been advocated for the treatment of depression. There are certain limitations to the use of CRF antagonists for the treatment of depression.

Accordingly, in one embodiment of the invention, the neurotransmitter system is the CRF system which includes corticotropin releasing factor as a neurotransmitter, the type of receptor is CRF receptors, the ligand is a CRF receptor agonist, and the counteradaptation causes a down-regulation of the CRF system. Regulation of the CRF system via counteradaptation is described in U.S. Provisional Patent Application Ser. No. 60/777,190, entitled “METHOD OF REGULATING THE CRF AND AVP SYSTEMS BY INDUCING COUNTERADAPTATIONS,” and filed on Feb. 27, 2006.

The CRF receptor may be, for example, CRF-1 or CRF-2. CRF-1 is generally associated with mood, while CRF-2 is generally associated with memory.

CRF agonists have effects on both the HPA and the extra-hypothalamic systems. With respect to the HPA, a CRF agonist causes the release of ACTH and beta endorphin into the circulation. In the near-term setting (i.e., during the first time period associated with each administration), the release of beta endorphin is beneficial because endorphins are known to improve hyperactive CRF conditions, such as depression and anxiety. But the CRF agonist would also increase ACTH release, which would have the effect of increasing cortisol release in the near-term. The elevation of cortisol is not what is needed when the goal is to depress the activity of a hyperactive CRF system. Furthermore, the agonist directly acts on the extra-hyphthalamic CRF system. Together, the effects on the ACTH release and on extra-hypothalamic CRF and AVP systems result in the worsening of the hyper-active CRF state in the near term. However, according to the present invention, the method described herein cause a counteradaptation, and an overall down-regulation of the CRF system, for example through down-regulation of ACTH relase and down-regulation of the extra-hypothalamic CRF system.

The counteradaptation may be a decrease in the biosynthesis or release of corticotropin releasing factor by the hypothalamus; a decrease in the number of the CRF receptors and/or binding sites on the CRF receptors; a decrease in the sensitivity of the receptors to binding by CRF receptor agonists and/or corticotropin releasing factor; or any combination thereof.

A variety of compounds may be used as CRF receptor agonists. For example, a number of peptide-based compounds are CRF receptor agonists. Examples include analogs of corticotrophin releasing factor and pharmaceutically acceptable salts and derivatives thereof. Cortagine is a suitable CRF receptor agonist for use in the methods of the present invention, described in Tezval, et al, PNAC, 101(25) (2004) and Todorovic, C., et al., Neurosci Biobehav Rev., 2005, 29(8): 1323-1333, each of which is incorporated herein by reference. Examples of CRF receptor agonists are described further in U.S. Pat. Nos. 5,132,111; 5,278,146; 5,824,771; 5,844,074; 6,214,797; 6,670,371; 6,812,210; 6,953,838, each of which is hereby incorporated by reference herein.

The initial dosage of the CRF receptor agonist is desirably high enough to induce a counteradaptive effect, but not so high as to cause the patient intolerable direct effects. For example, the initial dosage may be about 0.1 to 100 μg/kg/day initial dose and 100 to 1000 μg/kg/day for 8 hours slow release. hi certain embodiments of the invention, the initial dosage of the CRF receptor agonist is between about 1 to 50 μg/kg/day initial dose and 20 to 50 μg/kg/day for 8 hours slow release.

Desirably, neither a CRF receptor antagonist nor an AVP receptor antagonist is administered during the first time period associated with each administration of the CRF receptor agonist. In certain embodiments of the invention, however, a CRF receptor antagonist and/or an AVP receptor antagonist is administered during one or more of the second time periods associated with each administration of the CRF receptor agonist.

Examples of CRF antagonists include R121919 (Zobel, J Psychiatr Res 2000 May-June; 34(3): 171-81), DMP696 (Maciag, Neuropsychopharmacol 2002; 26: 574-582), antalarmin (Willenberg, Mol Psychiatry 2000 March; 5(2): 137-41), CP-154,526 (Mansbach, Eur J Pharmacol 1997 Mar. 26; 323(1): 21-6), SSR125543A (Briebel, J Pharmacol Exp Ther 2002 April; 301(1): 333-45), 2-arylamino-4-trifluoromethyl-aminomethylthiazole antagonists (Dubowchik, Bioorg Med Chem Lett 2004 Nov. 17; 13(22): 3997-4000), and astressin (Spina, Neuropsychopharmacol 2000; 22: 230-239), and pharmaceutically acceptable salts, analogues and derivatives thereof.

Examples of AVP receptor antagonists include peptidic AVP receptor antagonists such as d(CH2)5Tyr(Me)AVP, Phaa-d-Tyr(Me)-Phe-Gln-Asn-Arg-Pro-Arg-Tyr-NH₂, [Lys(3N₃ Phpa)⁸]HO-LVA, [d(CH2)5, D-Ile2, Ile4]-AVP, and [125I]-d(CH2)5[D-Tyr(Et)2, Val4, Tyr-NH29] AVP, and any analog of the AVP peptide (Cys-Tyr-Phe-Gln-Asn-Cys-Pro-Arg-Gly) that has AVP antagonist activity. AVP peptide antagonists may further include any pro-peptide molecule that is enzymatically cleaved into an active AVP antagonist. Examples of AVP receptor antagonists also include non-peptidic species such as OPC-21268 (1-(1-[4-(3-acetylaminopropoxy)benzoyl]-4-piperidyl)-3,4-dihydro-2(1H)-quinolinone), R 49059 (V1a antagonist), OPC-31260 (5-dimethylamino-1-[4-(2-methylbenzoylamino)benzoyl]-2,3,4,5-tetra hydro-1H-benzazepine), conivaptan (YM087—a V1a and V2 antagonist), Vaprisol (conivaptan) (V1a and V2 antagonist), VPA 985 (V2 antagonist), and YM 471 [(Z)-4′-[4,4-difluoro-5-[2-(4-dimethylaminopiperidino)-2-oxoethylidene]-2,3,4,5-tetrahydro-1H-1-benzoazepine-1-carbonyl]-2-phenylbenzanilide monohydrochloride]. AVP receptor antagonists are also described in U.S. Pat. Nos. 6,627,649 and 6,495,542, each of which is hereby incorporated by reference in its entirety.

In another embodiment of the invention, a mu and/or delta opiate receptor antagonist is administered in combination with the CRF agonist during the first time period with a ratio of administration half-life to the period between administrations no greater than ½. Administration methods for mu and/or delta opiate receptor antagonists are described hereinabove. This may be desirable due to the fact that the use of a CRF agonist, although intended to cause a down regulation of the CRF system, may have the unintended consequence of also down regulating the release of beta endorphin from the anterior pituitary gland. Such a reduced endorphin release would induce the opposite effect as to what is needed for the improvement of undesirable conditions that correlate inversely with the levels of circulating endorphins, such as depression and anxiety disorders. The repeated administration of the opiate antagonist may have two important effects: to block the endorphin down regulation by the CRF agonist, and also to cause an up-regulation of the endorphin system as described above. The principles for the administration of an opiate antagonist in order to induce an up regulation of the endogenous endorphin system are as described hereinabove. As described above, it may also be desirable to administer a CRF and/or AVP antagonist during the second time period associated with each administration of the CRF agonist. Desirably, neither a CRF receptor antagonist nor an AVP receptor antagonist is administered during the first time period associated with each administration.

CRF agonist administration according to this embodiment of the invention may be used to address an undesirable mental, neurological, or physiological condition in a patient, the undesirable mental, neurological or physiological condition being positively linked to CRF receptors. Examples of undesirable mental, neurological and physiological conditions that are positively linked to CRF receptors and addressable using this method include: melancholic depression, insufficient memory and a need for increased memory anticipated to occur in the future, anxiety and anxiety-related disorders, poor appetite and undereating disorders such as anorexia and bulimia, stress and stress that is anticipated to occur in the future, post-traumatic stress disorder, and a lack of motivation due to learning or memory problems. In desirable embodiments of the invention, the down-regulation of the CRF system causes a therapeutic benefit with respect to an undesirable mental, neurological or physiological condition positively linked to CRF receptors.

Intermittent administration of a CRF receptor agonist (with or without intermittent administration of a mu and/or delta opiate receptor antagonist) may be useful in improving an individual's ability to deal with a stressful situation, when such a situation should arise in the future. This is explained by the fact that small amounts of intermittent stress are actually good for a living organism. Such small amounts of intermittent stress stimulate the proper adaptations which allow that organism to better be able to deal with stressful situations when they arise in the future. Intermittent stress actually results in an intermittent increase in cortisol levels. Such an intermittent increase in cortisol levels is also what happens with the intermittent administration of a CRF and/or AVP agonist. Intermittent administration of a CRF receptor agonist thus mimics that which occurs with small amounts of intermittent stress. It induces a physiogical stress (i.e., increased CRF release) in the individual on a near-term temporary basis, which is a small enough ‘sress’ and of a short enough duration that it does not allow the side effects that can occur with chronic large stresses (i.e., hyperactive CRF and/or AVP systems). The method is intended to allow an individual to better be able to deal with a larger stress when it does occur in the future. In other words, a living organism that is subjected to a small amount of intermittent stress (i.e., temporarily increased CRF levels) will be better able to deal with a large amount of stress (i.e., large increase in CRF levels) in the future should such a situation arise, as compared to an organism that is never subjected to such short term temporary stress. When an acute stressful situation arises, it may be desirable to administer a CRF receptor antagonist and/or an AVP receptor antagonist during the second time period associated with each administration.

In another embodiment of the invention, the neurotransmitter system is the CRF system which includes corticotropin releasing factor as a neurotransmitter, the type of receptor is CRF receptors, the ligand is a CRF receptor antagonist, and the counteradaptation causes a up-regulation of the CRF system. Regulation of the CRF system via counteradaptation is described in U.S. Provisional Patent Application Ser. No. 60/777,190, entitled “METHOD OF REGULATING THE CRF AND AVP SYSTEMS BY INDUCING COUNTERADAPTATIONS,” and filed on Feb. 27, 2006.

The CRF receptor may be, for example, CRF-1 or CRF-2. CRF-1 is generally associated with mood, while CRF-2 is generally associated with memory.

The counteradaptation may be an increase in the biosynthesis or release of corticotropin releasing factor by the hypothalamus; an increase in the number of the CRF receptors and/or binding sites on the CRF receptors; an increase in the sensitivity of the receptors to binding by CRF receptor agonists and/or corticotropin releasing factor; or any combination thereof.

Suitable CRF antagonists are described hereinabove. The initial dosage of the CRF receptor antagonist is desirably high enough to induce a counteradaptive effect, but not so high as to cause the patient intolerable direct effects. For example, the initial dosage may be about 0.1 to 100 μg/kg/day initial dose and 100 to 1000 μg/kg/day for 8 hours slow release. In certain embodiments of the invention, the initial dosage of the CRF receptor antagonist is between about 1 to 50 μg/kg/day initial dose and 20 to 50 μg/kg/day for 8 hours slow release.

Desirably, neither a CRF receptor agonist nor an AVP receptor agonist is administered during the first time period associated with each administration of the CRF receptor antagonist. In certain embodiments of the invention, however, a CRF receptor agonist and/or an AVP receptor agonist is administered during one or more of the second time periods associated with each administration of the CRF receptor antagonist. Suitable CRF receptor agonists and AVP receptor agonists are described hereinabove.

In another embodiment of the invention, a mu and/or delta opiate receptor antagonist is administered in combination with the CRF antagonist during the first time period with a ratio of administration half-life to the period between administrations no greater than ½. Administration methods for mu and/or delta opiate receptor antagonists and the benefits thereof are described hereinabove.

CRF antagonist administration according to these embodiments of the invention may be used to address an undesirable mental, neurological, or physiological condition in a patient, the undesirable mental, neurological or physiological condition being negatively linked to CRF receptors. Examples of undesirable mental, neurological and physiological conditions that are negatively linked to CRF receptors and addressable using this method include, for example, atypical depression, weight gain and overeating disorders, lethargy and fatigue. In desirable embodiments of the invention, the up-regulation of the CRF system causes a therapeutic benefit with respect to an undesirable mental, neurological or physiological condition negatively linked to CRF receptors.

### The AVP System

AVP is a hormone, also called ADH (antidiuretic hormone) that is a nona-peptide (Cys-Tyr-Phe-Gln-Asn-Cys-Pro-Arg-Gly) with a cyclic structure, formed through a disulfide bridge between the two cysteine residues. AVP is the active hormone that is initially synthesized as a propeptide, ppAVP, which is 164 amino acids in length. The biologically active portion is AVP(20-28). The AVP nonapeptide is in certain instances further broken down into smaller biologically active fragments, ie., AVP(4-9), AVP(4-8), AVP(5-9), AVP 5-8). These smaller fragments lack peripheral endocrine activity, but do demonstrate selective activity within the CNS.

AVP hormone is produced by the hypothalamus, just as is CRF (and several other relasing factors). AVP is, however, produced from different secretory bodies than those which produce the releasing factors such as CRF (which flow directly to the anterior pituitary gland). The posterior pituitary gland is actually an extension of the hypothalamus. The AVP-producing cell bodies are in the hypothalamus. From the cell bodies the AVP is transported to the axonal terminals of these neuronal cell bodies, which is located in the posterior pituitary. From the posterior pituitary the AVP is released into the circulation.

In the circulation the AVP has two basic actions, peripheral and central. The peripheral actions pertain to vasoconstriction, glycogen metabolism and antidiuresis. The central actions pertain to learning and memory, social behaviors, thermoreguloation, autonomic function and mood.

AVP acts synergistically with CRF to stimulate the release of ACTH from the anterior pituitary. AVP also has direct effects on the adrenals for the release of cortisol. AVP has a similar effect on beta endorphin as does CRF, it stimulates beta endorphin release from the anterior pituitary gland.

AVP plays a role in stress and thus in depression and anxiety. Just as certain mood disorders (ie., depression and anxiety) are associated with a hyper-active CRF system, depression and anxiety are also associated with a hyper-active AVP system. AVP antagonists acting at the V1b receptor have demonstrated early favorable results as potential antidepressant and anxiolytic agents in animal studies. In addition, antagonists acting at the V1a receptor may also play a role as antidepressants and anxiolytics.

Accordingly, in one embodiment of the invention, the neurotransmitter system is the AVP system which includes corticotropin releasing factor as a neurotransmitter, the type of receptor is AVP receptors, the ligand is an AVP receptor agonist, and the counteradaptation causes a down-regulation of the AVP system. Regulation of the AVP system via counteradaptation is described in U.S. Provisional Patent Application Ser. No. 60/777,190, entitled “METHOD OF REGULATING THE CRF AND AVP SYSTEMS BY INDUCING COUNTERADAPTATIONS,” and filed on Feb. 27, 2006.

The AVP receptors may be, for example, V1R (also known as V1a), V2R or V3R (also known as V1b). V3R is the primary receptor in the pituitary. V1R is primarily in the liver and the brain, while V2R is primary in the kidney. In desirable embodiments of the invention, the AVP receptors are V1R receptors or V3R receptors.

The counteradaptation may be a decrease in the biosynthesis or release of arginine vasopressin by the hypothalamus; a decrease in the number of the AVP receptors and/or binding sites on the AVP receptors; a decrease in the sensitivity of the receptors to binding by AVP receptor agonists and/or arginine vasopressin; or any combination thereof.

A variety of compounds may be used as AVP receptor agonists. For example, a number of peptide-based compounds are AVP receptor agonists. Peptides may by synthetic or from mammal sources, such as bovine or porcine or others. Peptides may be linear or cyclic. Suitable peptidic AVP agonists include, for example, felypressin (2-L-Phe-8-L-lys AVP), desmopressin (1-(30mercaptopropionic acid)-8-D-AVP) and any peptide analog of the AVP peptide (Cys-Tyr-Phe-Gln-Asn-Cys-Pro-Arg-Gly) that has AVP agonist activity. AVP peptide agonists may further include any pro-peptide molecule that is enzymatically cleaved into an active AVP agonist. AVP peptide agonists may further include any compounds that contain the smaller biologically active fragments of the AVP nonapeptide, such as AVP(4-9), AVP(4-8), AVP(5-9), AVP (5-8). Other AVP receptor agonists suitable for use in the present invention include those disclosed in U.S. Pat. Nos. 6,090,803; 6,096,735; 6,096,736; 6,194,407; 6,235,900; 6,268,360; 6,297,234; 6,335,327; 6,344,451; 6,620,807; 6,642,223; and 6,831,079, each of which is incorporated herein by reference. As the skilled artisan will appreciate, pharmaceutically acceptable salts or derivatives of the above-described AVP agonists may also be used in the methods of the present invention.

The initial dosage of the AVP receptor agonist is desirably high enough to induce a counteradaptive effect, but not so high as to cause the patient intolerable direct effects. For example, the initial dosage may be about 0.1 to 100 μg/kg/day initial dose and 100 to 1000 μg/kg/day for 8 hours slow release. In certain embodiments of the invention, the initial dosage of the AVP receptor agonist is between about 1 to 50 μg/kg/day initial dose and 20 to 50 μg/kg/day for 8 hours slow release.

Desirably, neither a CRF receptor antagonist nor an AVP receptor antagonist is administered during the first time period associated with each administration of the CRF receptor agonist. In certain embodiments of the invention, however, a CRF receptor antagonist and/or an AVP receptor antagonist is administered during one or more of the second time periods associated with each administration of the CRF receptor agonist. CRF receptor antagonists and AVP receptor antagonists are described hereinabove.

In another embodiment of the invention, a mu and/or delta opiate receptor antagonist is administered in combination with the AVP agonist during the first time period with a ratio of administration half-life to the period between administrations no greater than ½. Administration methods for mu and/or delta opiate receptor antagonists are described hereinabove. This may be desirable due to the fact that the use of an AVP agonist, although intended to cause a down regulation of the AVP system, may have the unintended consequence of also down regulating the release of beta endorphin from the anterior pituitary gland. Such a reduced endorphin release would induce the opposite effect as to what is needed for the improvement of undesirable conditions that correlate inversely with the levels of circulating endorphins, such as depression and anxiety disorders. The repeated administration of the opiate antagonist may have two important effects: to block the endorphin down regulation by the AVP agonist, and also to cause an up-regulation of the endorphin system as described above. The principles for the administration of an opiate antagonist in order to induce an up regulation of the endogenous endorphin system are described hereinabove. As described above, it may also be desirable to administer a CRF and/or AVP antagonist during the second time period associated with each administration of the AVP agonist. Desirably, neither a CRF receptor antagonist nor an AVP receptor antagonist is administered during the first time period associated with each administration.

AVP agonist administration according to these embodiments of the invention may be used to address an undesirable mental, neurological, or physiological condition in a patient, the undesirable mental, neurological or physiological condition being positively linked to AVP receptors. Examples of undesirable mental, neurological and physiological conditions that are positively linked to AVP receptors and addressable using this method include: melancholic depression, insufficient memory and a need for increased memory anticipated to occur in the future, anxiety and anxiety-related disorders, poor appetite and undereating disorders such as anorexia and bulimia, stress and stress that is anticipated to occur in the future, post-traumatic stress disorder, and a lack of motivation due to learning or memory problems. In desirable embodiments of the invention, the down-regulation of the AVP system causes a therapeutic benefit with respect to an undesirable mental, neurological or physiological condition positively linked to AVP receptors.

Intermittent administration of a AVP receptor agonist (with or without intermittent administration of a mu and/or delta opiate receptor antagonist) may be useful in improving an individual's ability to deal with a stressful situation, when such a situation should arise in the future. This is explained by the fact that small amounts of intermittent stress are actually good for a living organism. Such small amounts of intermittent stress stimulate the proper adaptations which allow that organism to better be able to deal with stressful situations when they arise in the future. Intermittent stress actually results in an intermittent increase in cortisol levels. Such an intermittent increase in cortisol levels is also what happens with the intermittent administration of an AVP receptor agonist. Intermittent administration of an AVP receptor agonist thus mimics that which occurs with small amounts of intermittent stress. It induces a physiogical stress (i.e., increased AVP release) in the individual on a near-term temporary basis, which is a small enough ‘sress’ and of a short enough duration that it does not allow the side effects that can occur with chronic large stresses (i.e., hyperactive CRF and/or AVP systems). The method is intended to allow an individual to better be able to deal with a larger stress when it does occur in the future. In other words, a living organism that is subjected to a small amount of intermittent stress (i.e., temporarily increased AVP levels) will be better able to deal with a large amount of stress (i.e., large increase in AVP levels) in the future should such a situation arise, as compared to an organism that is never subjected to such short term temporary stress. When an acute stressful situation arises, it may be desirable to administer a CRF receptor antagonist and/or an AVP receptor antagonist during the second time period associated with each administration.

In another embodiment of the invention, the neurotransmitter system is the AVP system which includes corticotropin releasing factor as a neurotransmitter, the type of receptor is AVP receptors, the ligand is an AVP receptor antagonist, and the counteradaptation causes a up-regulation of the AVP system. Regulation of the AVP system via counteradaptation is described in U.S. Provisional Patent Application Ser. No. 60/777,190, entitled “METHOD OF REGULATING THE CRF AND AVP SYSTEMS BY INDUCING COUNTERADAPTATIONS,” and filed on Feb. 27, 2006.

The AVP receptors may be, for example, V1R (also known as V1a), V2R or V3R (also known as V1b). V3R is the primary receptor in the pituitary. V1R is primarily in the liver and the brain, while V2R is primary in the kidney. In desirable embodiments of the invention, the AVP receptors are V1R receptors or V3R receptors.

The counteradaptation may be an increase in the biosynthesis or release of corticotropin releasing factor by the hypothalamus; an increase in the number of the AVP receptors and/or binding sites on the AVP receptors; an increase in the sensitivity of the receptors to binding by AVP receptor agonists and/or arginine vasopressin; or any combination thereof.

Suitable AVP antagonists are described hereinabove. The initial dosage of the AVP receptor antagonist is desirably high enough to induce a counteradaptive effect, but not so high as to cause the patient intolerable direct effects. For example, the initial dosage may be about 0.1 to 100 μg/kg/day initial dose and 100 to 1000 μg/kg/day for 8 hours slow release. In certain embodiments of the invention, the initial dosage of the AVP receptor antagonist is between about 1 to 50 μg/kg/day initial dose and 20 to 50 μg/kg/day for 8 hours slow release.

Desirably, neither a CRF receptor agonist nor an AVP receptor agonist is administered during the first time period associated with each administration of the AVP receptor antagonist. In certain embodiments of the invention, however, a CRF receptor agonist and/or an AVP receptor agonist is administered during one or more of the second time periods associated with each administration of the CRF receptor antagonist. Suitable CRF receptor agonists and AVP receptor agonists are described hereinabove.

AVP antagonist administration according to this embodiment of the invention may be used to address an undesirable mental, neurological, or physiological condition in a patient, the undesirable mental, neurological or physiological condition being negatively linked to AVP receptors. Examples of undesirable mental, neurological and physiological conditions that are negatively linked to AVP receptors and addressable using this method include, for example, atypical depression, weight gain and overeating disorders, lethargy and fatigue, In desirable embodiments of the invention, the up-regulation of the AVP system causes a therapeutic benefit with respect to an undesirable mental, neurological or physiological condition negatively linked to AVP receptors.

In another embodiment of the invention, a mu and/or delta opiate receptor antagonist is administered in combination with the AVP antagonist during the first time period with a ratio of administration half-life to the period between administrations no greater than ½. Administration methods for mu and/or delta opiate receptor antagonists and the benefits thereof are described hereinabove.

### Immune-System Related Conditions

The methods of the present invention are useful in addressing and/or treating immune system-related conditions which are linked to receptors of the type of receptors of the neurotransmitter system. In these methods, the immune system is up-regulated. Examples of immune system-related conditions that may be treated or addressed using the methods of the present invention include: cancer, especially cancers having cancer cells substantially free of zeta receptors; autoimmune disorders, congenital immune deficiency, immune deficiencies due to immunosuppressant therapy, and acquired immune deficiencies. Examples of particular autoimmune disorders include: rheumatoid arthritis, multiple sclerosis, systemic lupus erythematosus, Addison's disease, ALS (Lou Gehrig's Disease), Alzheimer's Disease, Ankylosing Spondylitis, Autism Spectrum Disorders, Autoimmune Hemolytic Anemia, Autoimmune Progesterone Dermatitis, Behcet's Disease, Celiac Disease, Chronic Fatigue Syndrome, Churg-Strauss (allergic granulomatosis), CREST syndrome, Crohn's Disease, Dermatomyositis, Diabetes Mellitus, Emphysema (COPD), Endometriosis, Fibromyalgia, Goodpasture's disease, Graves disease, Hashimoto's thyroiditis, Interstitial Granulomatous Dermatitis, Irritable Bowel Syndrome (MS), Mixed Connective Tissue Disease, autoimmune-related connective tissue disorders, Myasthenia Gravis, Parkinson's Disease, Pemphigoid, Pernicious Anemia, Primary Lateral Sclerosis (PLS), Polychondritis (Relapsing), Polymyalgia Rheumatica, Polymyositis, Psoriasis, Sarcoidosis, Scleroderma, Sjogren's syndrome,Transverse Myelitis, Ulcerative Colitis, Vasculitis, Wegener's Granulomatosis, and autoimmune disorders secondary to infectious processes, administration of vaccines or environmental reactions (e.g., to chemicals such as silicones). According to one embodiment of the invention, the condition addressed by the method is the likelihood of becoming infected with an infectious microbe, such as a bacteria, fungus, parasite, mycobacteria, yeast, Chlamydia, protazon, helminth or rickettsia, or a virus such as, for example, HIV, influenza hepatitis (A,B,C), respiratory syncitial virus (RSV), gastrointestinal viral infections, encephalitis, and myocarditis. According to another embodiment of the invention, the immune system-related condition is the administration of a vaccine, and the up-regulation of the immune system results in the increased development of antibodies to an agent targeted by the vaccine.

In especially desirable embodiments of the invention, the neurotransmitter system is the endogenous endorphin system, and the type of receptor is delta opiate receptors, which are generally negatively linked to undesirable immune system-related conditions. The ligand is a delta opiate receptor antagonist, and the counteradaptation causes an up-regulation of the immune system. Any of the delta opiate receptor antagonists described hereinabove may be useful in this embodiment of the invention. In some embodiments of the invention, the delta opiate receptor antagonist is not naltrexone or naloxone. In some desirable embodiments of the invention, the initial dosage of the delta opiate receptor antagonist is greater than 10 mg/administration; greater than 10.5 mg/administration; greater than 11 mg/administration; or even greater than 15 mg/administration. The delta receptor antagonist is desirably a delta receptor selective antagonist; it desirably has substantially less activity with respect to mu receptors. One example of a suitable delta receptor antagonist has the structure:

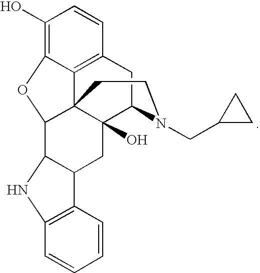

As described above with respect to mood, there may be a decrease in immune system function during the first time periods associated with each administration of the ligand. Further, if a method analogous to that described with reference to FIG. 6 is performed, then there may be a decrease in immune system function during the initial period of continuous dosing. Accordingly, it may be desirable to administer one or more additional medications during these periods. For example, it may be desirable to administer an autoimmune medication in combination with the ligand during the first time period and/or during a period of continuous ligand dosing. Examples of suitable autoimmune therapy include medications such as corticosteroids, chlorambucil, cyclosporine, cyclophosphamide, methotrexatate, azathioprine, TNF [symbol] antagonists, and therapies such as systemic enzyme therapy, gene therapy and irradiation therapy. Of course, as the skilled artisan will realize, other autoimmune medications may be used in the methods of the present invention, including those developed in the future. In certain embodiments of the invention, autoimmune therapy is not administered during the second time period.

Similarly, it may be desirable to administer an antiviral agent in combination with the ligand during the first time period and/or during a period of continuous ligand dosing. Examples of suitable antiviral agents include interferon, ribavirin, protease inhibitors, amantadine, rimantadine, pleconaril, antibodies (monoclonal, anti-VAP, receptor anti-idiotypic, extraneous receptor and synthetic receptor mimics), acyclovir, zidovudine (AZT), lamivudine, RNAase H inhibitors, integrase inhibitors, attachment blockers of transcription factors to viral DNA, so-called ‘antisense’ molecules, synthetic ribozymes, zanamivir, and osletamivir. Of course, as the skilled artisan will realize, other antiviral medications may be used in the methods of the present invention, including those developed in the future. In certain embodiments of the invention, the antiviral agent is not administered during the second time period.

Similarly, it may be desirable to administer an antimicrobial agent, an antifungal agent, and/or an antineoplastic agent in combination with the ligand during the first time period and/or during a period of continuous ligand dosing. In certain embodiments of the invention, the antimicrobial agent, the antifungal agent, and/or the antineoplastic agent is not administered during the second time period.

It may be desirable to administer an anti-cancer agent in combination with the ligand during the first time period and/or during a period of continuous ligand dosing. Suitable anti-cancer agents include, for example, adriamycin, busulfan, carboplatin, chlorambucil, cisplatin, cyclophosphamide, ifosfamide, mechlorethamine, melphalan, procarbazine, temozolamide, daunorubicin, doxorubicin, idarubicin, bleomycin, mitomycin, mitoxantrone, plicamycin, cytarabine fluorouracil, hydroxyurea, methotrexate, asparaginase, pegaspargase, irinotecan, topotecan, bicalutamide, estramustine, flutamide, leuprolide, megestrol, nilutamide, testosterone, triptorelin, anastrazole, letrozole, aldesleukin, alemtuzumab, gemtuzumab, toremifene, trastuzumab, etoposide, docetaxel, paclitaxel, vinblastine, vincristine, vinorelbine, altretamine, Erlotinib, gleevec, curcumin, tamoxifen, bortezomib, gefitinib, imatinib, cancer cell growth inhibitors derived from 3,4-methylenedioxy-5,4′-dimethoxy-3′-amino-Z-stilbene, hydroxyphenstatin and its sodium diphosphate prodrug, histone deacetylase inhibitors, suberoylanilide hydroxamic acid, trichostatin A, sodium butyrate, metformin, five-lipoxygenase (5-LO) antagonists, antisense oligonucleotides targeting the RI [symbol] regulatory subunit of protein kinase A type I, Vitamin E and its analogs, vitamin E succinate (VES), and gene therapy. Of course, as the skilled artisan will realize, other anti-cancer agents may be used in the methods of the present invention, including those developed in the future. In certain embodiments of the invention, the antiviral agent is not administered during the second time period.

Cancer is one undesirable immune-related condition that may be treated and/or addressed using the methods described herein. In one desirable embodiment of the invention, up-regulation of the immune system is achieved by up-regulating the endogenous endorphin system using repeated administration of delta opiate receptor antagonists as described hereinabove in order to treat or address cancer. Both cancer cells and the immune cells (i.e., killer cells) have opiate receptors. When the immune system undergoes an up regulation, the killer cells are given an enhanced ability to ‘attack’ cancer cells. A further benefit is that the up-regulated cancer cells are made more susceptible to cell death. Desirably, the methods described with reference to FIG. 6 are used, in which a continuous dose of ligand is first given to induce up-regulation of the immune system in a relatively quick manner. The time for this continuous dose could range, for example, from one day to several weeks or months depending on the severity of the cancer.

In addition to merely blocking the enhanced cancer cell proliferation by the anti-cancer agents there is an added advantage to the simultaneous use of an opiate antagonist with an anti-cancer agent. Because the cancer cells are induced to grow more rapidly they are made more susceptible to the effects of anti-cancer agents. Anti-cancer agents are thus able to have an enhanced toxic effect on cancer cells during this period of time that the cancer cells are rapidly proliferating. The anti-cancer agents are in this way made to be more efficacious in inducing cancer cell death.

The methods described in the present invention may also be used to treat and/or address immune disorders and infectious diseases. In one desirable embodiment of the invention, up-regulation of the immune system is achieved by up-regulating the endogenous endorphin system using repeated administration of delta opiate receptor antagonists as described hereinabove in order to treat or address cancer. In a manner similar to initiating cancer therapy, one may opt to begin treatment with a temporary period of continuous receptor blockade, as described above with reference to FIG. 6.

The counteradaptative response to, for example, opiate antagonism is the up regulation of the immune system. An up regulated immune system results in an enhanced immune response by such immune cells as killer cells as well as other immune cells. Such an up regulated immune system can be utilized to treat conditions that have an abnormal immune system. These immune disorders can be selected from, but not limited to, autoimmune disorders, congenital immune deficiencies, immune deficiencies due to immunosuppressant therapy (ie., for cancer, transplantation), or acquired immune deficiencies (ie., AIDS). An up regulated immune system can be of further benefit in that it is intended to enhance the body's ability to fight off infections, which include bacterial and viral infections, as well as those from other infectious microbes.

Autoimmune disorders are those that have an abnormally functioning immune system wherein an immune response is generated against the body's own tissues. Because autoimmune disorders are the result of the immune system turning up its activity against normal cells and cellular components, it would appear that, if the immune system were enhanced, then it should worsen such disorders, due to the enhanced activity against one's own tissues. On the contrary, autoimmune system disorders have been shown to respond favorably to an enhanced immune system.

One embodiment of the present invention relates to using relatively high doses (ie., an equivalency of greater than 10 mg naloxone or naltrexone, which further includes numerous analogs) of delta opiate antagonists for an even relatively strong up-regulation of the endogenous endorphin system, hence a relatively strong up-regulation of the immune system.

For many infections there is a secondary added benefit achieved when practicing methods analogous to those described with reference to FIG. 6, in which there is an initial period of continuous receptor blockade. For example, with a viral infection, during this period of receptor blockade there would be an enhanced proliferation of the viral particles, due to the suppression of the immune system. One could take advantage of this by administering agents that kill or inhibit the virus during replication. Because the viral particles are proliferating at a more rapid pace they would become more susceptible to agents that inhibit their growth and lead to their death. In this way the antiviral agent is made more potent because it attacks the virus during replication when it is vulnerable. It is of further benefit in that, following the period of continuous receptor blockade, there is an up regulated immune system, wherein the immune cells are more potent in destroying any remaining viral particles. Such concomitant antiviral agents may be selected from the group interferon, ribavirin, any one of a number of protease inhibitors, amantadine, rimantadine, pleconaril, antibodies (monoclonal, anti-VAP, receptor anti-idiotypic, extraneous receptor and synthetic receptor mimics), acyclovir, zidovudine (AZT), lamivudine, RNAase H inhibitors, integrase inhibitors, attachment blockers of transcription factors to viral DNA, ‘antisense’ molecules, synthetic ribozymes, zanamivir (Relenza®), and osletamivir (Tamiflu®). As the skilled artisan will appreciate, any suitable antiviral medication may be used.

The methods of the present invention may be used in a prophylactic manner, i.e., to lessen the likelihood of becoming infected with any potential infectious microbe or virus. The methods of the present invention may be performed, for example, before an operation to lessen the risk of infection during and after the operation. The methods of the present invention may be used as a prophylactic measure against virtually any infectious agent, such as HIV, hepatitis, influenza, RSV, tuberculosis, protozoa, rickettsia, malaria and staphylococcus.

### Other Conditions

According to another aspect of the present invention, the methods described hereinabove are used to address or treat cardiovascular or lipid or cholesterol metabolism-related conditions, such as cardiovascular disorders (e.g., cardiac, peripheral vascular and stroke). The methods of the present invention are also useful in treating or addressing diabetes (e.g., type I and type II).

Disorders of lipid or cholesterol metabolism generally are predisposing to CV and diabetic disorders. Lipid or cholesterol metabolism-related disorder that may be addressed or treated using the methods of the present invention include hypertriglyceridemia, hypercholesterolemia, (including generalized elevation of cholesterol and/or elevation of low-density-lipoprotein cholesterol [LDL], or abnormally low levels of high-density-lipoprotein [HDL] cholesterol). Disorders of lipid metabolism further include the lipodystrophies that are due to HIV (AIDS virus) infections.

As described above, the AVP neurotransmitter system is involved in stress, the ACTH/cortisol pathway and mood disorders such as depression and anxiety. A different and very specific type of stress is called ‘nutrient stress’. Nutrient stress is a stress situation that occurs with fasting, starvation, or insulin-induced hypoglycemia. Nutrient stress results in elevated glucocorticoid levels (cortisol). However, unlike classic stress, CRF release does not play a major role in nutrient stress cortisol release. Instead, AVP is the primary modulator of cortisol release in response to nutrient stress.

Nutrient stress is critical when one discusses factors that are related to extending life. It was discovered 70 years ago that caloric restriction (which induces nutrient stress) can extend an organism's life by about 30%. This phenomenon has since been demonstrated to also occur in numerous mammals including the rat, mouse, dog and possibly the primate. In fact, caloric restriction is the only method that has been proven to be capable of increasing an organism's lifespan.

Caloric restriction is thought to work by activating what is called the SIR 2 gene in yeast, which is known as SIRT1 in mammals. The SIRT1 gene encodes an enzyme Sirt1, which targets proteins that control such critical processes as apoptosis (cell death), cell defenses and metabolism. Sirt1 is thus thought to be the master controller of a regulatory system for aging that is activated by stress.

For example, Sirt1 is a central metabolic regulator in the liver, muscle and fat cells. This indicates that Sirt1 is involved in fat storage, and in this way it is linked to aging and metabolic diseases such as type 2 diabetes. Another critical process that is modified by Sirt1 is inflammation. Caloric restriction is known to suppress excessive inflammation, which is associated with aging processes such as heart disease and neurodegeneration. Sirt1 further regulates production of IGF-1 (insulin-like growth factor). IGF-1 is known to dictate life span in various organisms, -worms, flies, mice, and possibly humans.

Compounds that modulate the activity of Sir2 and its human cousins are collectively referred to as ‘Sirtuins’. Sirtuin-activating compounds may be abbreviated ‘STAC’. One such STAC is reservatrol. It is commonly found in red wine and in a variety of plants when they are stressed. In fact, some of the beneficial effects on health from red wine are thought to be due to reservatrol, and or other STACs. There are numerous additional (at least 18) compounds which are produced by plants in response to stress that modulate sirtuins.

One aspect of the invention relates to methods in which the physiologic control mechanisms that result from caloric restriction are mimicked in order to cause an activation of the Sirt1 pathway. Any of the methods and of the present invention result in intermittent stress being put on a patient; accordingly, any of the methods of the present invention will be useful in activating the Sirt1 pathway, and therefore to treat or address undesirable conditions linked to the Sirt1 pathway, such as conditions related to aging, fat metabolism, inflammation and the IGF-1 system. Such conditions include, for example, cancer, arthritis, asthma, heart disease and neurodegeneration. The methods of the present invention may also be used as a prophylactic measure against physiological changes caused by aging. Any of the neurotransmitter systems described above may be used by the skilled artisan to activate the Sirt1 pathway.

Caloric restriction is one manner of inducing what has been termed a ‘hormetic’ effect, or hormesis. In 1904, Starling coined the word “hormone” to designate any substance that is produced in small amounts, and is then carried in blood to influence some other organ. It is from the Greek “Hormo,” meaning, “To excite.” Southam and Erlich found that high concentrations of oak bark extract inhibited fungal growth, but in low dose it stimulated fungal growth. (Phytopathology 33:517, 1943). They modified Starling's word to “Hormesis” which describes the notion that small doses of a toxin can be helpful. Hormesis is further generalized as a term that refers to the long term benefits of mild, repeated stress or stimulation.

One characteristic of hormesis is that it can be activated following a certain stimulus. Mild stress such as increased external temperature, mild radiation exposure, or hypergravity, as well as nutritional stress (i.e. Caloric Restriction, [Frame, L. T., et al., “Caloric Restriction as a Mechanism Mediating Resistance to Environmental Disease”, Environ Health Perspect, 1998, 106 (Suppl 1): 303-324], all have been shown to improve a range of parameters associated with aging. Because caloric restriction is not easily attainable there has been a drive to develop compounds which mimic the effects of caloric restriction. Such compounds are referred to as ‘caloric restriction mimetics’ (Weindruch, R., et al., “Caloric Restriction Mimetics—Metabolic Interventions”, Journals of Gerontology Series A: Biological Sciences and Medical Sciences, 2001, 56: 20-33.).

It is thus not only caloric restriction that induces physiologic parameters that improve age related conditions. Any low degree of intermittent stress can also induce physiologic parameters that act as defense mechanisms. In this respect, any mild intermittent stress will be beneficial for an organism as it induces physiological parameters that give it an improved host defense mechanism.

According to one aspect of the invention, intermittent dosing of neurotransmitter receptor ligands (as described herein above) is used to induce a mild intermittent stress, which in turn activates defense mechanisms that protect the organism for times of stress. Any neurotransmitter system linked to dysphoria may be used to mimic caloric restriction using the methods described hereinabove. For example, all of the neurotransmitter systems described hereinabove are linked to the stressful condition of dysphoria. Accordingly, all of the methods, neurotransmitter systems and ligands described hereinabove may be used to induce a minor transient stress, and therefore can be used to induce hormetic effects and mimic the effects of caloric restriction. In certain embodiments of the invention, these defense mechanisms mechanisms that protect the organism for times of stress in order to improve conditions of aging, fat metabolism, inflammation and the IGF-1 system such as cancer, arthritis, asthma, heart disease and neurodegeneration.

Athletic performance is associated with various neurotransmitter systems, such as the endogenous endorphin systems. The body produces endorphins in response to exercise. The level of intensity and the level of athletic training is directly associated with the endorphin response. (see Mougin, et al, Eur J App Physiol, 1988; Doiron, et al, J Str Cond Res, 1999; Sforzo, Sport Med, 1989: and Golbfarb, et al., Sport Med, 1997.) Because highly trained athletes and high levels of intensity during exercise are directly related to endorphin release, it would be of benefit for improving athletic performance with an up regulated endorphin system. Accordingly, one aspect of the present invention relates to the use of the methods, neurotransmitter systems and ligands described hereinabove for improving athletic performance. In addition, just as a temporary period of continous receptor blockade may be beneficial in cancer therapy, treatment infectious diseases and autoimmune therapy as described above, the skilled artsian may use a temporary period of continuous receptor blockade, for example as described with reference to FIG. 6, during athletic training. According to one embodiment of the present invention, the neurotransmitter system is the endogenous endorphin system, the ligand is a mu and/or delta opiate receptor antagonist, and the counteradaptation is an up-regulation of the endogenous endorphin system.

Cognition (e.g., learning and memory) is also directly associated with neurotransmitter systems such as the endogenous endorphin system. (Riley, et al., Neurosci Biobehav Rev, 1980; Getsova, et al., Neurosci Behav Physiol, Biomed & Life Sci & Russian Library of Sci, 1986.) The acute administration of a delta opiate receptor antagonist (e.g., naltrexone) inhibits learning and memory. (Chaves, et al., Neuropsychologia, 1988). The link between neurotransmitter systems and cognition is further indicated by the fact that levels of glucocorticoids in the circulation are related to cognitive function; persistently high levels of cortisol are associated with impaired memory. (Li, et al., Neurobio Aging, 2005). On the other hand, transient elevation of glucocorticoids improves learning and memory tasks. (Patel, et al., Neurol Aging, 2002). Accordingly, one aspect of the present invention relates to the use of the methods, neurotransmitter systems and ligands described hereinabove to improve cognition. For example, these methods may be used to cause only a transient elevation of glucocorticoids. Use of any of the methods, neurotransmitter systems, and ligands described hereinabove will cause minor, intermittent stress, which in turn causes transient temporary elevations of glucocorticoids. Accordingly, any of the methods, neurotransmitter systems and ligands described hereinabove can be used to improve cognition. In another example, to improve cognition the skilled artisan can effect an up-regulation of the endogenous system using a mu and/or delta opiate receptor antagonist as described hereinabove. In addition, just as a temporary period of continous receptor blockade may be beneficial in cancer therapy, treatment of infectious diseases and autoimmune therapy as described above, the skilled artsian may use a temporary period of continuous receptor blockade, for example as described with reference to FIG. 6, in order to create a faster improvement of cognitive function.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents. All references cited herein are hereby incorporated by reference in their entirety.

1-374. (canceled)

375. A method of regulating a neurotransmitter system by inducing a counteradaptation in a patient, the neurotransmitter system including a type of receptor linked to an inflammatory condition, the method comprising the step of:
repeatedly administering to the patient a ligand for the type of receptor linked to an inflammatory condition, each administration having an administration half-life, thereby causing the ligand to bind receptors of that type during a first time period associated with each administration and inducing a counteradaptation,
wherein (i) the counteradaptation causes the regulation of the neurotransmitter system, and alleviation of the inflammatory condition;
(ii) the ratio of the administration half-life to the period between administrations is no greater than ½; and
(iii) the ligand becomes unbound to the type of receptor during a second time period associated with each administration.

376. The method according to claim 375, wherein the regulation of the neurotransmitter system causes a therapeutic benefit with respect to the inflammatory condition linked to the type of receptor.

377. The method according to claim 375, wherein the ligand is an agonist for receptors of the type of receptor, and the regulation is a down-regulation of the neurotransmitter system.

378. The method of claim 377, wherein the inflammatory condition is positively linked to the type of receptor.

379. The method of claim 377, wherein an antagonist for receptors of the type of receptor is not administered during the first time period associated with each administration.

380. The method of claim 377, wherein each administration has a second time period associated therewith, the second time period being subsequent to the first time period associated with the administration, and wherein an antagonist for receptors of the type of receptor is administered during one or more of the second time periods.

381. The method according to claim 375, wherein each first time period is at least about five minutes in duration.

382. The method according to claim 375, wherein each first time period is less than about twenty four hours in duration.

383. The method according to claim 375, wherein the ratio of the administration half-life to the period between administrations is no greater than ⅓.

384. The method according to claim 375, wherein the ratio of the administration half-life of the ligand to the period between administrations is greater than 1/24.

385. The method according to claim 375, wherein the dose of ligand at each administration is increased over time.

386. The method according to claim 375, wherein the dose of ligand at each administration is increased intermittently over time.

387. The method according to claim 375, wherein the administration of the ligand is performed daily.

388. The method according to claim 375, wherein the administration half-life is less than about four, eight or sixteen hours.

389. The method according to claim 375, wherein the administration half-life is greater than about four hours.

390. The method according to claim 375, wherein the compound half-life of the ligand is less than about sixteen hours.

391. The method according to claim 375, wherein the compound half-life of the ligand is greater than about twelve hours, and wherein the method further comprises
administering repeatedly and with a period of less than every two days a second ligand for the type of receptor, each administration of the second ligand having an administration half-life of less than about eight hours.

392. The method according to claim 375, wherein the administration is repeated at least five times.

393. The method according to claim 375, wherein a substantial fraction of the first time period occurs while the patient is asleep.

394. The method according to claim 375, wherein each administration of the ligand is performed within the hour before the patient goes to bed.

395. The method according to claim 375, further comprising:
administering an anxiolytic agent, a hypnotic agent, autoimmune therapy, an antiviral agent, an antimicrobial agent, an antifungal agent, an antineoplastic agent, or an anti-cancer agent in combination with the ligand.

396. The method according to claim 375, wherein the method is used to address or treat a cardiovascular or lipid or cholesterol metabolism-related condition in a patient in need thereof.

397. The method according to claim 375, wherein the method is used to address or treat an inflammatory condition in a patient in need thereof.
